SEQUENCE LISTING (48 sequences)

SEQ ID NO: 1 - DNA, 19 bp, Artificial Sequence
  Synthetic
  misc_feature (1)..(19): SEQ ID NO 13
gaaagttacaacctccgcg

SEQ ID NO: 2 - DNA, 20 bp, Artificial Sequence
  Synthetic
  misc_feature (1)..(20): SEQ ID NO 14
gatgttggcgaacattggcg

SEQ ID NO: 3 - DNA, 20 bp, Artificial Sequence
  Synthetic
  misc_feature (1)..(20): SEQ ID NO 15
gaacctctgcctctcggtag

SEQ ID NO: 4 - DNA, 20 bp, Artificial Sequence
  Synthetic
  misc_feature (1)..(20): SEQ ID NO 16
gatactgcggcccgttggga

SEQ ID NO: 5 - DNA, 19 bp, Artificial Sequence
  Synthetic
  misc_feature (1)..(19): SEQ ID NO 17
gacatccaccgtaagcgca

SEQ ID NO: 6 - DNA, 20 bp, Artificial Sequence
  Synthetic
  misc_feature (1)..(20): SEQ ID NO 18
gaagagaccactcaggaaca

SEQ ID NO: 7 - DNA, 20 bp, Artificial Sequence
  Synthetic
  misc_feature (1)..(20): SEQ ID NO 19
gcgcttacggtggatgtcct

SEQ ID NO: 8 - DNA, 20 bp, Artificial Sequence
  Synthetic
  misc_feature (1)..(20): SEQ ID NO 20
gctccctttcgcgttcgctg

SEQ ID NO: 9 - DNA, 19 bp, Artificial Sequence
  Synthetic
  misc_feature (1)..(19): SEQ ID NO 21
gacgtgaccgcaatgaacg

SEQ ID NO: 10 - DNA, 20 bp, Artificial Sequence
  Synthetic
  misc_feature (1)..(20): SEQ ID NO 22
gtatgtagtctgcgatgacg

SEQ ID NO: 11 - DNA, 20 bp, Artificial Sequence
  Synthetic
  misc_feature (1)..(23): SEQ ID NO 23
gtcttcactgtccgccatgt

SEQ ID NO: 12 - DNA, 20 bp, Artificial Sequence
  Synthetic
  misc_feature (1)..(20): SEQ ID NO 24
ggagttcaagatgatctgcg

SEQ ID NO: 13 - DNA, 20 bp, Artificial Sequence
  Synthetic
  misc_feature (1)..(20): SEQ ID NO 25
gaagctcaccaataacgcac

SEQ ID NO: 14 - DNA, 20 bp, Artificial Sequence
  Synthetic
  misc_feature (1)..(20): SEQ ID NO 26
gtacgtcctgtagggcggct

SEQ ID NO: 15 - DNA, 20 bp, Artificial Sequence
  Synthetic
  misc_feature (1)..(20): SEQ ID NO 27
ggaatcaccggctccgggca

SEQ ID NO: 16 - DNA, 20 bp, Artificial Sequence
  Synthetic
  misc_feature (1)..(20): SEQ ID NO 28
gtggacatggtaccagaaga

SEQ ID NO: 17 - DNA, 19 bp, Artificial Sequence
  Synthetic
  misc_feature (1)..(19): SEQ ID NO 29
gacgacgtgagccgaaagc

SEQ ID NO: 18 - DNA, 19 bp, Artificial Sequence
  Synthetic
  misc_feature (1)..(19): SEQ ID NO 30
gatgggggcaagtacggta

SEQ ID NO: 19 - DNA, 20 bp, Artificial Sequence
  Synthetic
  misc_feature (1)..(20): SEQ ID NO 31
ggctcacgtcgtctcgagtg

SEQ ID NO: 20 - DNA, 20 bp, Artificial Sequence
  Synthetic
  misc_feature (1)..(20): SEQ ID NO 32
ggcaaacacgtgcaaattct

SEQ ID NO: 21 - DNA, 20 bp, Artificial Sequence
  Synthetic
  misc_feature (1)..(20): SEQ ID NO 33
gagctccagtggtgtcgcga

SEQ ID NO: 22 - DNA, 19 bp, Artificial Sequence
  Synthetic
  misc_feature (1)..(19): SEQ ID NO 34
gacgggtgtgacagactct

SEQ ID NO: 23 - DNA, 19 bp, Artificial Sequence
  Synthetic
  misc_feature (1)..(19): SEQ ID NO 35
gatcgtcgcaggtcggatc

SEQ ID NO: 24 - DNA, 20 bp, Artificial Sequence
  Synthetic
  misc_feature (1)..(20): SEQ ID NO 36
gacacgtggcatccagatct

SEQ ID NO: 25 - DNA, 20 bp, Artificial Sequence
  Synthetic
  misc_feature (1)..(20): SEQ ID NO 37
gtaaaggcaacgataaaccc

SEQ ID NO: 26 - DNA, 20 bp, Artificial Sequence
  Synthetic
  misc_feature (1)..(20): SEQ ID NO 38
gacggatccgcaccagcagg

SEQ ID NO: 27 - DNA, 20 bp, Artificial Sequence
  Synthetic
  misc_feature (1)..(20): SEQ ID NO 39
gattgcggcgtggcggtcag

SEQ ID NO: 28 - DNA, 20 bp, Artificial Sequence
  Synthetic
  misc_feature (1)..(20): SEQ ID NO 40
gttccacctgggcttctcag

SEQ ID NO: 29 - DNA, 19 bp, Artificial Sequence
  Synthetic
  misc_feature (1)..(19): SEQ ID NO 41
gagaacgtacacgagtatc

SEQ ID NO: 30 - DNA, 20 bp, Artificial Sequence
  Synthetic
  misc_feature (1)..(20): SEQ ID NO 42
gatctgccagacgcggatgg

SEQ ID NO: 31 - DNA, 20 bp, Artificial Sequence
  Synthetic
  misc_feature (1)..(20): SEQ ID NO 43
gagctcgtggagctggaagg

SEQ ID NO: 32 - DNA, 20 bp, Artificial Sequence
  Synthetic
  misc_feature (1)..(20): SEQ ID NO 44
gggagagactctgtttcacc

SEQ ID NO: 33 - DNA, 19 bp, Artificial Sequence
  Synthetic
  misc_feature (1)..(19): SEQ ID NO 45
gagcacttgtccccgaaaa

SEQ ID NO: 34 - DNA, 19 bp, Artificial Sequence
  Synthetic
  misc_feature (1)..(19): SEQ ID NO 46
gaacgtggttcggttccgc

SEQ ID NO: 35 - DNA, 20 bp, Artificial Sequence
  Synthetic
  misc_feature (1)..(20): SEQ ID NO 47
gatatcgtcaacattcggga

SEQ ID NO: 36 - DNA, 20 bp, Artificial Sequence
  Synthetic
  misc_feature (1)..(20): SEQ ID NO 48
ggcaccgcgcttgatctgta

SEQ ID NO: 37 - DNA, 20 bp, Artificial Sequence
  Synthetic
  misc_feature (1)..(20): SEQ ID NO 49
gcgaaagcaacagtatgaat

SEQ ID NO: 38 - DNA, 19 bp, Artificial Sequence
  Synthetic
  misc_feature (1)..(19): SEQ ID NO 50
gtacgcggaggcaatatcg

SEQ ID NO: 39 - DNA, 20 bp, Artificial Sequence
  Synthetic
  misc_feature (1)..(20): SEQ ID NO 51
gattagtcggtgcgcgtgaa

SEQ ID NO: 40 - DNA, 20 bp, Artificial Sequence
  Synthetic
  misc_feature (1)..(20): SEQ ID NO 52
ggagcatgtacgagttgctg

SEQ ID NO: 41 - DNA, 20 bp, Artificial Sequence
  Synthetic
  misc_feature (1)..(20): SEQ ID NO 53
gatccgcctgtagtctcgca

SEQ ID NO: 42 - DNA, 20 bp, Artificial Sequence
  Synthetic
  misc_feature (1)..(20): SEQ ID NO 54
gacgggcagtctagcgtgtc

SEQ ID NO: 43 - DNA, 57 bp, Artificial Sequence
  Synthetic
  misc_feature (1)..(57): SEQ ID NO 55
  misc_feature (1)..(1): /5BiosG/
cgtaatacgactcactatagggcttcagccaaggaagctacatttaggtgcactaag

SEQ ID NO: 44 - DNA, 60 bp, Artificial Sequence
  Synthetic
  misc_feature (1)..(1): /5BiosG/
  misc_feature (1)..(60): SEQ ID NO 56
gctagttattgctcagcgggtcttgtttctcggtgtgcttgctatttctagctctaaaac

SEQ ID NO: 45 - DNA, 60 bp, Artificial Sequence
  Synthetic
  misc_feature (1)..(60): SEQ ID NO 57
gtgtaaaacgacggccagtatggcaccaactcgatgacgtaatacgactcactatagggc

SEQ ID NO: 46 - DNA, 60 bp, Artificial Sequence
  Synthetic
  misc_feature (1)..(60): SEQ ID NO 58
caggaaacagctatgacatagtcctgctgtaccaggcgtctgctagttattgctcagcgg

SEQ ID NO: 47 - DNA, 43 bp, Artificial Sequence
  Synthetic
  misc_feature (1)..(43): SEQ ID NO 59
  misc_feature (18)..(20): N can be any nucleotide residue, e.g., any of A, G, C. or T
atttaggtgacactatannngttttagagctagaaatagcaag

SEQ ID NO: 48 - DNA, 80 bp, Artificial Sequence
  Synthetic
  misc_feature (1)..(80): SEQ ID NO 60
aaaagcaccgactcggtgccactttttcaagttgataacggactagccttattttaacttgctatttctagctctaaaac

(19) United States
(12) Patent Application Publication
(10) Pub. No.: US 2024/0287609 A1
(43) Pub. Date: Aug. 29, 2024

(54) COMPOSITIONS AND METHODS FOR LARGE-SCALE IN VIVO GENETIC SCREENING

(71) Applicant: UNIVERSITY OF UTAH RESEARCH FOUNDATION, Salt Lake City, UT (US)

(72) Inventors: Saba Parvez, Salt Lake City, UT (US); Randall T. Peterson, Salt Lake City, UT (US); Jing-Ruey Joanna Yeh, Winchester, MA (US)

(21) Appl. No.: 18/568,690

(22) PCT Filed: Jun. 8, 2022
(86) PCT No.: PCT/US2022/032704

§ 371 Date: Dec. 8, 2023

Related U.S. Application Data
(60) Provisional application No. 63/251,826, filed on Oct. 4, 2021.
(60) Provisional application No. 63/208,399, filed on Jun. 8, 2021.

(51) Int. Cl. C12Q 1/6883; C12N 9/22; C12N 15/11; C12Q 1/44; C12Q 1/6806; C12Q 1/6869

(52) U.S. Cl. CPC C12Q 1/6883; C12N 9/22; C12N 15/11; C12Q 1/44; C12Q 1/6806; C12Q 1/6869; C12N 2310/20; C12N 2310/30; C12Q 2600/158

(57) ABSTRACT

Disclosed herein are droplets comprising gene editing systems and barcodes. The disclosure further relates to methods for large-scale identification of genes in vivo using barcodes and methods for large-scale identification of gene function in a plurality of subjects using a plurality of droplets.

### CROSS-REFERENCE TO RELATED APPLICATIONS

This application claims priority to U.S. Provisional Patent Application No. 63/208,399, filed Jun. 8, 2021 and U.S. Provisional Patent Application No. 63/251,826, filed Oct. 4, 2021, each of which is incorporated herein by reference in its entirety.

### STATEMENT REGARDING FEDERALLY SPONSORED RESEARCH

This invention was made with government support under grant GM134069 awarded by the National Institutes of Health. The government has certain rights in the invention.

### REFERENCE TO SEQUENCE LISTING

This application is filed with a Computer Readable Form of a Sequence Listing in accord with 37 C.F.R. § 1.821(c). The text file submitted by EFS, “U-7251-026389-9322-WO01-SEQ-LIST_ST25.txt,” was created on Jun. 7, 2022, has a file size of 12.5 Kilobytes, and is hereby incorporated by reference in its entirety.

### FIELD

This disclosure relates to droplets comprising gene editing systems and barcodes. The disclosure further relates to methods for large-scale identification of genes in vivo using barcodes and methods for large-scale identification of gene function in a plurality of subjects using a plurality of droplets.

### INTRODUCTION

Historically, large scale genetic screens in zebrafish have employed forward genetic techniques such as chemical or insertional mutagenesis. These screens have proven invaluable in identifying key pathways regulating vertebrate development and behavior. While impressive in scale, forward genetic techniques are time- and labor-intensive requiring years to link a desired phenotype with the genotype.

Reverse genetics approaches such as Clustered Regularly Interspaced Short Palindromic Repeats (CRISPR) have potential to circumvent some of the issues of forward genetics but are severely limited in throughput. Targeting genes-of-interest is typically done one gene at a time-designing individual guide RNAs (gRNA), injecting Cas9-gRNA ribonucleoprotein (RNP) complexes, maintaining, propagating, and genotyping groups of subjects such as fish-requiring extensive time, labor, and space. The largest such screen to date targeted 128 genes in zebrafish. Recent studies used multiplexed gRNAs to generate biallelic F0 mutants that successfully phenocopy germline mutant phenotypes, but have not been scaled up for genome-wide genetic screens. CRISPR-Cas9 can be scaled up for large-scale screens in cultured cells, but CRISPR screens in animals have been challenging because generating, validating, and keeping track of large numbers of mutant animals is prohibitive.

Thus, there is a need for methods of large-scale functional genetic screening in vivo that provide efficient identification of genes responsible for morphological or behavioral phenotypes.

### SUMMARY

In an aspect, the disclosure relates to a water-in-oil droplet that may comprise: an aqueous phase may comprise a gene editing system and a barcode oligonucleotide; and an oil phase may comprise an oil and a surfactant; wherein the aqueous phase may be encapsulated by the oil phase. In an embodiment, the gene editing system may be a Clustered Regularly Interspaced Short Palindromic Repeats-CRISPR associated proteins (CRISPR-Cas) system, a transcription activator like effector nuclease (TALEN) system, or a zinc finger nuclease (ZFN) system. In another embodiment, the oil may be 3M™ Novec™ 7500, Bio-Rad Droplet Generation Oil for Probes, or a polysiloxane. In another embodiment, the oil phase comprises from about 90% to about 99.9% of the oil. In another embodiment, the surfactant may be 008-Fluorosurfactant, Pico-Surf™, or a dendronized fluorosurfactant. In another embodiment, the oil phase comprises from about 0.1% to about 10% of the surfactant.

In a further aspect, the disclosure relates to a method for large-scale identification of a gene in vivo in a plurality of subjects, the method may comprise: administering to the plurality of subjects a plurality of barcode oligonucleotides; isolating one or more barcode oligonucleotides from one or more subjects from the plurality of subjects that exhibit one or more phenotypes of interest; amplifying the isolated barcode oligonucleotides; and, sequencing the amplified barcode oligonucleotides. In an embodiment, the barcode oligonucleotides comprise an end-cap modification at the 5′ end of the oligonucleotide. In another embodiment, the end-cap modification may be biotinylation, 2′OMe, or phosphorothioate. In another embodiment, the barcode oligonucleotide may be unmodified. In another embodiment, the plurality of subjects are highly prolific organisms. In another embodiment, the highly prolific organisms are fish, insects, or worms.

Another aspect of the disclosure provides a method for large-scale identification of gene function in a plurality of subjects, the method may comprise: administering to the plurality of subjects a plurality of water-in-oil droplets may comprise: an aqueous phase may comprise a gene editing system and one or more barcode oligonucleotides; and an oil phase, wherein the aqueous phase may be encapsulated by the oil phase; isolating the one or more barcode oligonucleotides from one or more subjects from the plurality of subjects that exhibit one or more phenotypes of interest; amplifying the isolated one or more barcode oligonucleotides; and, sequencing the amplified one or more barcode oligonucleotides. In an embodiment, the oil phase comprises an oil and a surfactant. In another embodiment, the oil may be 3M™ Novec™ 7500, Bio-Rad Droplet Generation Oil for Probes, or a polysiloxane. In another embodiment, the oil phase comprises from about 90% to about 99.9% of the oil. In another embodiment, the surfactant may be 008-Fluorosurfactant, Pico-Surf™, or a dendronized fluorosurfactant. In another embodiment, the oil phase comprises from about 0.1% to about 10% of the surfactant. In another embodiment, the gene editing system may be a Clustered Regularly Interspaced Short Palindromic Repeats-CRISPR associated proteins (CRISPR-Cas) system, a transcription activator like effector nuclease (TALEN) system, or a zinc finger nuclease (ZFN) system. In another embodiment, the one or more barcode oligonudeotides comprise an end-cap modification at the 5′ end of the oligonucleotide that prevents exonuclease and endonuclease degradation of the one or more barcode oligonucleotides. In another embodiment, each subject of the plurality of subjects may be administered one water-in-oil droplet from the plurality of water-in-oil droplets that comprises a gene editing system that targets a different gene in each subject. In another embodiment, the plurality of water-in-oil droplets are administered to the plurality of subjects simultaneously.

The disclosure provides for other aspects and embodiments that will be apparent in light of the following detailed description and accompanying figures.

### BRIEF DESCRIPTION OF THE DRAWINGS

FIG. 1 is a schematic showing a DNA barcode produced by extending and adding a 5′-Biotin group to the DNA template used for in vitro transcription.

FIG. 2 is a schematic showing production of a DNA barcode for sequencing with M13F or M13R primers.

FIGS. 3A-3D show that MIC-Drop enables high-throughput CRISPR screens in zebrafish. FIG. 3A is a workflow of the MIC-Drop platform. A microfluidics device generates nanoliter-sized droplets, each containing ribonucleoproteins (RNP) targeting a gene-of-interest and a unique DNA barcode associated with the gene. Droplets targeting multiple genes are intermixed, loaded into a single injection needle and injected serially into one-cell zebrafish embryos. Embryos showing phenotypes-of-interest are isolated and the causative genotype is identified by retrieving and sequencing the barcode. FIG. 3B is a photograph showing droplets are uniform in size. Distance between bars is 0.1 mm. FIG. 3C is a series of photographs showing that injection of droplets containing RNPs targeting tyr, rx3, tbx5a, and chrd genes recapitulates known mutant phenotypes in F0, highlighted by boxes. FIG. 3D is a bar chart showing that RNP-containing droplets are non-toxic and stable for prolonged storage-retaining activity at least 28 days of storage at 4° C. a: Uninjected; b: Traditional RNP injection; c: MIC-Drop injection. FIG. 3E is a photograph of a single-needle comprising hundreds of intermixed, colored droplets (used as proxies for droplets targeting different genes) showing that the droplets do not fuse when transferred to an injection needle. FIG. 3F is a bar graph showing that there was an even representation of each droplet with a majority of embryos exhibiting only one of the three expected phenotypes in zebrafish embryos that were injected using a single-needle of intermixed droplets targeting three different genes (tyr, tnnt2a, chrd).

FIGS. 4A-4D show that multiplexed gRNA injection recapitulates mutant phenotypes in F0 embryos. FIG. 4A is a schematic comparing the advantages and disadvantages of forward-genetics vs reverse-genetics in zebrafish. MIC-Drop enables the targeted mutagenesis of reverse-genetics and the scalability of forward-genetics. FIGS. 4B-D show that injection of Cas9 and 4 gRNAs targeting each gene-of-interest recapitulates known mutant phenotypes in F0 embryos with no significant toxicity (FIG. 4C) and with high efficiency (FIG. 4D).

FIGS. 5A-5E show that MIC-Drop enables single-needle injection of droplets targeting multiple genes. FIGS. 5A-5B are bar charts showing that incorporation of DNA barcodes in the droplets does not alter viability of the injected embryos (FIG. 5A) but does cause a slight increase in deformities resulting from nucleic acid toxicity (FIG. 5B). FIGS. 5C-D are bar charts showing that single-needle injection of intermixed droplets targeting 3 genes (FIG. 5C) or 8 genes (FIG. 5D) and subsequent phenotyping and barcode sequencing reveal a proportionate representation of the droplets, with most embryos showing one of the unique phenotypes. About 5% of embryos show mixed phenotype and consequent mixed barcode sequencing results likely due to unintended co-injection of more than one droplet. FIG. 5E is a series of images of electrophoretic gels showing that the DNA barcodes are stable after injection in embryos and can be successfully retrieved and sequenced at 168 hpf (7dpf).

FIGS. 6A-6B show that multiplexed gRNA injection results in high targeted editing. FIG. 6A is a schematic showing that a T7E1 assay in embryos injected with multiplexed gRNAs targeting tyr gene reveals high editing efficiency. Amplicons from the targeted site show large deletions (top gel; tyr samples 1-6). Treatment of the amplicons with T7 endonuclease shows multiple bands (bottom gel) suggesting high indel frequencies in the injected embryos. FIG. 6B is a diagram showing amplicon sequencing of tnnt2a exon 3 in embryos injected with multiplexed gRNAs targeting tnt2a exon 3 reveals mosaicism with near complete editing efficiency and with a high frequency of 5-20 bp deletions in the targeted site.

FIGS. 7A-7D show that MIC-Drop enables large-scale phenotypic screens and small molecule target identification. Schematic of a spike-in (FIG. 7A) phenotypic and (FIG. 7B) behavioral screen to test robustness of the MIC-Drop platform. FIG. 7A shows for the phenotypic screen, droplets targeting either tyr or npas4/were intermixed with droplets containing non-targeting scrambled gRNAs (scr) in a 1:50 ratio. After single-needle droplet injection, the percentage of embryos showing albino or cloche phenotypes was scored. Inset shows the albino and cloche phenotypes are recovered at a frequency of ˜2%, which is the expected frequency from a 1:50 ratio mix. FIG. 7B is similar to FIG. 7A, except droplets targeting trpa1b were intermixed with scr droplets in a 1:20 ratio. Following injection, embryos were arrayed in a multi-well plate, treated with optovin, and assayed for light-dependent motor response. FIG. 7C shows images of traces tracking movement in zebrafish from embryos injected with droplets targeting trpa1b as compared to zebrafish from scramble-injected and non-injected embryos in response to optovin and light. White boxes around wells indicate wells that contain droplet-injected embryos that show little or no movement upon co-administration of optovin and violet light. The “+” signs indicate rows of embryos that were treated with optovin.

FIG. 7D shows the quantitation of the zebrafish movement tracking in FIG. 7C and reveals that embryos injected with droplets targeting trpa1b were refractory to optovin- and light-induced motion response.

FIGS. 8A-8D show that MIC-Drop enables identification of gene targets of small-molecules. FIGS. 8A-C show treatment of zebrafish embryos with optovin (+) results in a light-dependent motion response. Embryo tracking (FIG. 8A) and quantitation of movement (FIGS. 8B-C) shows increased zebrafish activity triggered by pulsed violet light. Embryos injected with a set of non-targeting scrambled gRNAs (bottom) behave the same as uninjected controls (top) (FIG. 8B). Embryos injected with gRNAs targeting trpa1b are refractory and show no light-triggered movement (FIG. 8A). Optovin- and light-triggered activity quantitation of three sample embryos injected with trpa 1b-targeting gRNAs. FIG. 8D shows diagnostic PCR used to test the barcode identities of embryos injected with 20:1 mix of droplets targeting scrambled: trpa 1b (also see FIG. 7C). 6.25% of the intermixed droplet-injected embryos (9/144) have the trpa1b barcode. Uninjected embryos were used as negative controls. Lines are drawn on top of gel bands for ease of viewing.

FIGS. 9A-9F show a proof-of-concept genetic screen to identify novel regulators of cardiovascular development. FIG. 9A shows data using a publicly available dataset to populate a list of candidate genes enriched in the embryonic zebrafish heart. About 14% of the genes (dots) have reported cardiac phenotypes in ZFIN suggesting enrichment of genes important in heart development. FIG. 9B is a schematic showing filtering to remove genes with known mutant phenotypes yields 192 poorly-characterized genes potentially important for cardiovascular development in zebrafish. FIG. 9C is a graph showing that gRNA sequences with less off-targets were primarily used. FIG. 9D is a series of bar charts showing that a MIC-Drop screen of the 188 candidate genes and subsequent phenotyping shows no significant differences in viability between uninjected and droplet-injected embryos by 3 dpf. Embryos with gross morphological defects at 3 dpf (˜15%) were removed and the barcodes of those with cardiac defects were sequenced. Droplets targeting npas4/were spiked-in at 2% proportion as positive control. FIG. 9E is a chart showing that barcode sequencing of embryos displaying cardiac phenotypes yields “hit” candidates. Heat map shows the observed frequency of each barcode. As positive controls, barcodes for tnnt2a, nkx2.5, and npas4/were enriched in embryos with cardiac phenotypes. Genes with barcode frequency of ≥4 (Binomial probability <0.05) or with consistent cardiac phenotypes were considered for secondary validation. FIG. 9F is a bar chart showing that secondary validation by direct RNP injection corroborates screening results and identifies a dozen novel genes, the loss of which results in cardiac phenotypes in at least 20% of F0 embryos.

FIGS. 10A-10B show RNAseq data analysis to curate a list of candidate genes important in vertebrate heart development. FIG. 10A shows a principle-component analysis (PCA) and a volcano plot of differentially expressed genes in the zebrafish heart vs. the zebrafish muscle tissue. FIG. 10B shows a PCA and a volcano plot of differentially expressed genes in the adult heart vs. the embryonic heart. PCA analysis shows high sample-to-sample concordance (3 samples of each). Highlighted dots on volcano plots show genes enriched in the heart relative to muscle and embryonic heart relative to adult heart. Horizontal line (5% FDR); vertical line (2-fold differential expression).

FIGS. 11A-11F show that CRISPR screen using MIC-Drop identifies novel genes responsible for cardiovascular development. FIG. 11A shows o-dianisidine staining shows loss of alad results in porphyria, which can be rescued by co-injection of alad mRNA. FIG. 11B shows loss of gstm.3 or atp6v1c1 results in abnormal cardiac electrophysiology. Isochronal maps and action potential measurements reveal reduced conduction velocities, and shorter ventricular action potential duration in the gstm.3 and atp6v1c1 crispants relative to uninjected controls. Loss of (FIG. 11C) actb2, (FIG. 11D) clec19a, (FIG. 11E) gse1, and (FIG. 11F) ppan result in distinct cardiac malformations. actb2 crispants have a small ventricle with reduced number of ventricular cardiomyocytes 1: Control; 2: actb2-targeting gRNAs (FIG. 11C). Loss of clec19a and gse1 result in abnormal morphogenesis and an extended atrioventricular canal relative to wildtype embryos (FIGS. 11D-E). Alcian blue staining of ppan crispants shows abnormal jaw and skull development, which is rescued by ppan mRNA injection. The embryos also display cardiac edema, and a silent ventricle (FIG. 11F).

FIGS. 12A-12E show that a CRISPR screen using MIC-Drop discovers novel genes responsible for vertebrate heart and blood development. FIG. 12A shows injection of alad mRNA rescues the porphyria phenotype of alad crispants (also see FIG. 11A). The number of embryos counted is reported above each bar. FIG. 12B shows representative action potential duration graphs of gstm.3 and atp6v1c1 crispants show shorter delay between atrium and ventricle beats compared to uninjected controls. FIG. 12C shows loss of atp6v1c1b alone recapitulates the phenotypes observed in crispants injected with gRNAs targeting both atp6v1c1a and atp6v1c1b ohnologs. Two gRNAs (1 and 2) were used per ohnolog. FIG. 12D shows, similarly, loss of actb2 alone results in cardiac defects. FIG. 12E shows the cardiac phenotype resulting from actb2 loss can be rescued with injection of actb2 mRNA.

FIGS. 13A-13D show that a CRISPR screen identifies novel genes responsible for cardiac development and function. FIG. 13A shows cox8a and ddah2 crispants display cardiac edema and incomplete cardiac looping. Black outline: ventricle; grey outline: atrium; atrium in the wild type (grey dashed line) is looped properly and therefore out of focus from the ventricle.

FIGS. 13B-C show loss of ppan results in cardiac edema, an abnormal heart, as well as jaw and craniofacial deformities. Alcian blue staining of 5 dpf embryos and quantitation (FIG. 13C) shows the deformities can be rescued by injection of ppan mRNA. FIG. 13D shows, similarly, various phenotypes including a bent trunk, head and eye deformities, and a silent ventricle in sf3b4 crispants can be completely rescued with sf3b4 mRNA injection.

FIG. 14 is a photograph of a DNA electrophoretic gel illustrating several DNA barcoding strategies. Unmodified and various end-modified DNA barcodes were injected in zebrafish embryos. 48 hours post-injection, the DNA barcodes were successfully amplified (amplicon of 215 base pair length) and sequenced, irrespective of the barcode modifications. Bio stands for biotin modification, PS stands for phosphorothioate modification of the first 3 nucleotides, 2′-O-Me stands for 2′-O-methyl RNA modification. All modified oligos were ordered from IDT.

FIGS. 15A-15B are graphs illustrating the stability of RNA barcodes. FIG. 15A shows that in vitro transcribed mRNA is stable for up to 36 hours post injection in zebrafish embryos, and can successfully reverse transcribed and amplified. FIG. 15B shows that in vitro transcribed gRNAs can be successfully captured, reverse-transcribed, and subsequently amplified for sequencing multiple days after injection.

### DETAILED DESCRIPTION

Described herein is a platform combining droplet microfluidics, single-needle en masse gene-editing system injections, and barcoding to enable large-scale functional genetic screens in a plurality of subjects. In one application, the droplet system can identify small molecule targets. Furthermore, the droplet system can be used to discover genes important for phenotypes in subjects. With the potential to scale to thousands of genes, the droplet system and methods described herein using the droplet system enables genome-scale reverse-genetic screens in model organisms.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by a gene editing system described herein such as a CRISPR/Cas-based gene editing system.

“Clustered Regularly Interspaced Short Palindromic Repeats” and “CRISPRs”, as used interchangeably herein, refer to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an organism to which the nucleic acid is administered. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used interchangeably. The reference level may be a predetermined value or range, which is employed as a benchmark against which to assess the measured result. “Control group” as used refers to a group of control organisms. The predetermined level may be a cutoff value from a control group. The predetermined level may be an average from a control group. The healthy or normal levels or ranges for a target or for a protein activity or phenotype may be defined in accordance with standard practice. A control may be a subject or cell without a gene editing system as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the subject to whom the nucleic acid molecule is administered. As used herein, the term “expressible form” refers to gene constructs that contain the necessary regulatory elements operable linked to a coding sequence that encodes a protein such that when present in the cell of the subject, the coding sequence will be expressed.

“Genome editing” or “gene editing” as used herein refers to changing a gene. Genome editing may include correcting or restoring a mutant gene or adding additional mutations. Genome editing may include knocking out a gene, such as a mutant gene or a normal gene. Genome editing may be used to treat disease by changing the gene of interest or to identify a gene of interest.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nudeotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or“operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. A motif may include 3, 4, 5, 6, or 7 sequential amino acids. A domain may be comprised of a series of the same type of motif.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “organism” as used herein interchangeably refers to any vertebrate or invertebrate, including, but not limited to, a subject that wants or is in need of the herein described compositions or methods. The subject may be a human or a non-human. The subject may be a highly proliferative organism such as a fish, insect, or worm. The subject may comprise a plurality of subjects such as embryos. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” or “gene of interest” as used herein refers to any nucleotide sequence encoding a known or putative gene product. The target gene may be a mutated gene involved in a genetic disease. In certain embodiments, the target gene is a gene whose function is unknown.

“Target region” or “target sequence” as used herein refers to the region of the target gene to which the gene editing or targeting system is designed to bind. The portion of the gene editing system, such as gRNA, that targets the target sequence in the genome may be referred to as the “targeting sequence” or “targeting portion” or “targeting domain.”

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome, or yeast artificial chromosome. A vector may be a DNA or RNA vector. A vector may be a self-replicating extrachromosomal vector, and preferably, is a DNA plasmid. For example, the vector may encode a gene editing system as described herein.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. DROPLET COMPOSITIONS

Provided herein are water-in-oil droplets. The water-in-oil droplets may include an aqueous phase and an oil phase. The aqueous phase comprises aqueous droplets. The oil phase comprises an oil carrier for delivery of the aqueous droplets. The aqueous phase may be encapsulated by the oil phase. The water-in-oil droplets may be formulated so as not to fuse together and so that their contents do not mix when multiple water-in-oil droplets are contained within the same container, such as a syringe. The total mass of one aqueous droplet may be about 1 μg.

The total volume of aqueous droplets and the total volume of oil in a container may vary based on how densely the droplets are packed together in the container. For example, the total volume in a container occupied by the aqueous phase may comprise less than 1% of the total volume of the container or the total volume in a container occupied by the aqueous phase may comprise greater than 50% of the total volume of the container. The aqueous phase may comprise a buffer, water, a dye such as phenol red, salts, water-soluble compounds such as glycerol and PEG, or a combinations thereof. The aqueous phase may comprise a gene editing system, a barcode oligonucleotide, or a combination thereof. The gene editing systems or barcode oligonucleotides as detailed herein, or at least one component thereof, may be formulated into the aqueous phase of the water-in-oil droplets in accordance with standard techniques well known to those skilled in the art. The aqueous phase can be formulated according to the type of gene editing system or barcode to be used. The aqueous phase of the water-in-oil droplets may be sterile, pyrogen free, and particulate free. An isotonic formulation may be used. Generally, additives for isotonicity may include sodium chloride, dextrose, mannitol, sorbitol and lactose. In some cases, isotonic solutions such as phosphate buffered saline may be used.

The total volume of aqueous droplets and the total volume of oil in a container may vary based on how densely the droplets are packed together in the container. For example, the total volume in a container occupied by the oil phase may comprise less than 50% of the total volume of the container or the total volume in a container occupied by the oil phase may comprise greater than 99% of the total volume of the container. The oil phase may comprise an oil and a surfactant. The oil phase may comprise from about 90% to about 99.9%, from about 91% to about 99.9%, from about 92% to about 99.9%, from about 93% to about 99.9%, from about 94% to about 99.9%, from about 95% to about 99.9%, from about 96% to about 99.9%, or from about 97% to about 99.9% of the oil. The oil may be any oil that allows for formation of stable water-in-oil droplets that do not readily fuse with each other, does not inactivate the components in the aqueous droplets (i.e. is inert), is biocompatible, and is non-toxic to a subject that is to be administered the water-in-oil droplet. For example, the oil may be a fluorinated oil. Another example of the oil may be 3-ethoxy-1, 1,1,2,3,4,4,5,5,6,6,6-dodecafluoro-2-trifluoromethyl-hexane (3M™ Novec™ 7500, also known as hydrofluoroether (HFE)-7500), Bio-Rad Droplet Generation Oil for Probes, or polysiloxanes (e.g., Laos and Benner, (2022) PLoS ONE 17(1): e0252361). The oil is not mineral oil, Halocarbon® oil 27, Novec™ 7000 Novec™ 7200, or Bio-Rad Droplet generation oil for EvaGreen®. The oil phase may comprise from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, or from about 0.1% to about 3% of the surfactant. The surfactant may be any surfactant that allows for formation of stable water-in-oil droplets that do not readily fuse with each other, is miscible with the oil, does not inactivate the components in the aqueous droplets (i.e. is inert), is biocompatible, and is non-toxic to a subject that is to be administered the water-in-oil droplet. For example, the surfactant may be a fluorosurfactant. Another example of the surfactant may be 008-Fluorosurfactant, Pico-Surf™, a dendronized fluorosurfactant (e.g., Chowdhury et al. (2019) Nat Commun. 10, 4546). The surfactant is not sorbitan monooleate such as Span™ 80, t-Octylphenoxypolyethoxyethanol such as Triton™ X-100, NP-40, or polysorbate 20 such as Tween®20.

### 3. GENE EDITING SYSTEMS

a. CRISPR/Cas9-Based Gene Editing System

The gene editing system of the present disclosure may include a CRISPR/Cas9-based gene editing system. In some embodiments, the water-in-oil droplets may comprise from about 10 pg to about 10 ng of gRNA(s) and from about 0.1 μM to about 150 UM of a Cas9 protein. In other embodiments, the water-in-oil droplets may comprise from about 1 μg to about 1 μg of DNA encoding the CRISPR/Cas-based gene editing system. The CRISPR/Cas9-based gene editing system may include a Cas9 protein or a fusion protein or DNA encoding the Cas9 protein or mRNA for synthesis of the Cas9 protein, and at least one gRNA or DNA encoding the at least one gRNA. The CRISPR/Cas9-based gene editing system may comprise from 1 to 10 gRNAs, from 1 to 9 gRNAs, from 2 to 8 gRNAs, from 3 to 7 gRNAs, from 4 to 6 gRNAs, or from 4 to 5 gRNAs that target the same gene. The CRISPR/Cas9-based gene editing system may comprise 4 gRNA that target the same gene. The concentration of the CRISPR/Cas9-based gene editing systems and buffers for supporting delivery of the CRISPR/Cas9-based gene editing systems are well established and known in the art.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas9 forms a complex with the 3′ end of the sgRNA (which may be referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and Ill effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9: crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements.

An engineered form of the Type II effector system of S. pyogenes was shown to function in eukaryotic cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing. The CRISPR/Cas9-based engineered systems can be designed to target any gene, including genes involved in, for example, a genetic disease. The CRISPR/Cas9-based gene editing system can include a Cas9 protein or a Cas9 fusion protein.

### i) Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., Ccycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobactereiseniae. In certain embodiments, the Cas9 molecule is a Streptococcus pyogenes Cas9 molecule (also referred herein as “SpCas9”).

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi: 10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi: 10.1038/nmeth.2681). In the aforementioned embodiments, N can be any nucleotide residue, for example, any of A, G, C, or T. Cas9 molecules can be engineered to alter the PAM specificity of the Cas9 molecule.

Additionally or alternatively, a nucleic acid encoding a Cas9 molecule or Cas9 polypeptide may comprise a nuclear localization sequence (NLS). Nuclear localization sequences are known in the art.

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonudease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. Exemplary mutations with reference to the S. aureus Cas9 sequence to inactivate the nuclease activity include D10A and N580A.

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein.

### ii) Cas9 Fusion Protein

Alternatively or additionally, the CRISPR/Cas9-based gene editing system can include a fusion protein. The fusion protein can comprise two heterologous polypeptide domains. The first polypeptide domain comprises a Cas9 protein or a mutated Cas9 protein. The first polypeptide domain is fused to at least one second polypeptide domain. The second polypeptide domain has a different activity that what is endogenous to Cas9 protein. For example, the second polypeptide domain may have an activity such as transcription activation activity, transcription repression activity, transcription release factor activity, histone modification activity, nuclease activity, nucleic acid association activity, methylase activity, or demethylase activity. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

iii) gRNA

The CRISPR/Cas-based gene editing system includes at least one gRNA molecule or “guide”. For example, the CRISPR/Cas-based gene editing system may include four gRNA molecules. The at least one gRNA molecule can bind and recognize a target region. The gRNA provides the targeting of a CRISPR/Cas9-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The CRISPR/Cas9-based gene editing system may include at least one gRNA, wherein the gRNAs target different DNA sequences. The target DNA sequences may be overlapping. The target DNA sequences may affect the same gene. The target sequence or protospacer is followed by a PAM sequence at the 3′ end of the protospacer in the genome. Different Type II systems have differing PAM requirements, as detailed above.

As described above, the gRNA molecule comprises a targeting domain (also referred to as targeted or targeting sequence), which is a polynucleotide sequence complementary to the target DNA sequence. The gRNA may comprise a “G” or a “GA” or a “GN” at the 5′ end of the targeting domain or complementary polynucleotide sequence. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, or at least 15 different gRNAs. The number of gRNA molecules that may be included in the CRISPR/Cas9-based gene editing system can be less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

### iv) Repair Pathways

The CRISPR/Cas9-based gene editing system may be used to introduce site-specific double strand breaks at targeted genomic loci. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

b. Transcription Activator Like Effector Nuclease (TALEN) System

The gene editing system of the present disclosure may include a TALEN-based gene editing system. The TALEN-based gene editing system may be designed to target any gene, for example, a gene involved in a genetic disease. The TALEN-based gene editing system may include a nuclease and a TALE DNA-binding domain that binds to the target gene, or DNA encoding the nuclease and the TALE DNA-binding domain, or mRNA for synthesis of the nuclease and TALE DNA-binding domain. In some embodiments, the water-in-oil droplets may comprise from about 0.1 μM to about 150 UM of the TALE DNA-binding domain and from about 0.1 μM to about 150 UM of the nuclease. In other embodiments, the water-in-oil droplets may comprise from about 1 μg to about 1 μg of DNA encoding the TALEN-based gene editing system. The concentration of the TALEN-based gene editing systems and buffers for supporting delivery of the TALEN-based gene editing systems are well established and known in the art.

A Transcription Activator-like Effector (TALE) is a protein that recognizes and binds to a particular DNA sequence. The DNA-binding domain of a TALE includes an array of tandem 33-35 amino acid repeats, also known as repeat-variable di-residue (RVD) modules. Each RVD module specifically recognizes a single base pair of DNA. RVD modules may be arranged in any order to assemble an array that recognizes a defined DNA sequence. The binding specificity of a TALE DNA-binding domain is determined by the RVD array followed by a single truncated repeat of, for example, 20 amino acids. A TALE DNA-binding domain may have an array of 1 to 30 RVD modules, each RVD module recognizing a single base pair of DNA. The TALE DNA-binding domain may have an RVD array length from 1-30 modules, from 1-25 modules, from 1-20 modules, from 1-15 modules, from 5-30 modules, from 5-25 modules, from 5-20 modules, from 5-15 modules, from 7-25 modules, from 7-23 modules, from 7-20 modules, from 10-30 modules, from 10-25 modules, from 10-20 modules, from 10-15 modules, from 15-30 modules, from 15-25 modules, from 15-20 modules, from 15-19 modules, from 16-26 modules, from 16-41 modules, from 20-30 modules, or from 20-25 modules in length. The RVD array length may be 5 modules, 8 modules, 10 modules, 11 modules, 12 modules, 13 modules, 14 modules, 15 modules, 16 modules, 17 modules, 18 modules, 19 modules, 20 modules, 22 modules, 25 modules, or 30 modules. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors and/or nucleases. In some embodiments, a TALE is fused to or includes a nuclease domain and may be referred to as a TALE nuclease (TALEN). The nuclease domain may include, for example, the endonuclease Fokl. TALENs may recognize target sites that consist of two TALE DNA-binding sites that flank a 12-bp to 20-bp spacer sequence recognized by the Fokl cleavage domain.

“Transcription activator-like effector nucleases” or “TALENs” as used interchangeably herein refers to engineered fusion proteins of the catalytic domain of a nuclease, such as endonuclease Fokl, and a designed TALE DNA-binding domain that may be targeted to a custom DNA sequence. A “TALEN monomer” refers to an engineered fusion protein with a catalytic nuclease domain and a designed TALE DNA-binding domain. Two TALEN monomers may be designed to target and cleave a target region.

TALENs may be used to introduce site-specific double strand breaks at targeted genomic loci. Site-specific double-strand breaks are created when two independent TALENs bind to nearby DNA sequences, thereby permitting dimerization of Fokl and cleavage of the target DNA. TALENs have advanced genome editing due to their high rate of successful and efficient genetic modification. This DNA cleavage may stimulate the natural DNA-repair machinery, leading to one of two possible repair pathways: homology-directed repair (HDR) or the non-homologous end joining (NHEJ) pathway.

In some embodiments, the number of TALE DNA-binding domains that may be included in the TALEN-based gene editing system can be at least 1 TALE DNA-binding domain, at least 2 different TALE DNA-binding domains, at least 3 different TALE DNA-binding domains, at least 4 different TALE DNA-binding domains, at least 5 different TALE DNA-binding domains, at least 6 different TALE DNA-binding domains, at least 7 different TALE DNA-binding domains, at least 8 different TALE DNA-binding domains, at least 9 different TALE DNA-binding domains, at least 10 different TALE DNA-binding domains, at least 11 different TALE DNA-binding domains, at least 12 different TALE DNA-binding domains, at least 13 different TALE DNA-binding domains, at least 14 different TALE DNA-binding domains, or at least 15 different TALE DNA-binding domains. The number of TALE DNA-binding domain molecules that may be included in the TALEN-based gene editing system can be less than 30 different TALE DNA-binding domains, less than 25 different TALE DNA-binding domains, less than 20 different TALE DNA-binding domains, less than 19 different TALE DNA-binding domains, less than 18 different TALE DNA-binding domains, less than 17 different TALE DNA-binding domains, less than 16 different TALE DNA-binding domains, less than 15 different TALE DNA-binding domains, less than 14 different TALE DNA-binding domains, less than 13 different TALE DNA-binding domains, less than 12 different TALE DNA-binding domains, less than 11 different TALE DNA-binding domains, less than 10 different TALE DNA-binding domains, less than 9 different TALE DNA-binding domains, less than 8 different TALE DNA-binding domains, less than 7 different TALE DNA-binding domains, less than 6 different TALE DNA-binding domains, less than 5 different TALE DNA-binding domains, less than 4 different TALE DNA-binding domains, less than 3 different TALE DNA-binding domains, or less than 2 different TALE DNA-binding domains. The number of TALE DNA-binding domains that may be included in the TALEN-based gene editing system can be between at least 1 TALE DNA-binding domain to at least 30 different TALE DNA-binding domains, at least 1 TALE DNA-binding domain to at least 25 different TALE DNA-binding domains, at least 1 TALE DNA-binding domain to at least 20 different TALE DNA-binding domains, at least 1 TALE DNA-binding domain to at least 16 different TALE DNA-binding domains, at least 1 TALE DNA-binding domain to at least 12 different TALE DNA-binding domains, at least 1 TALE DNA-binding domain to at least 8 different TALE DNA-binding domains, at least 1 TALE DNA-binding domain to at least 4 different TALE DNA-binding domains, at least 4 different TALE DNA-binding domains to at least 30 different TALE DNA-binding domains, at least 4 different TALE DNA-binding domains to at least 25 different TALE DNA-binding domains, at least 4 different TALE DNA-binding domains to at least 20 different TALE DNA-binding domains, at least 4 different TALE DNA-binding domains to at least 16 different TALE DNA-binding domains, at least 4 different TALE DNA-binding domains to at least 12 different TALE DNA-binding domains, at least 4 different TALE DNA-binding domains to at least 8 different TALE DNA-binding domains, 8 different TALE DNA-binding domains to at least 30 different TALE DNA-binding domains, at least 8 different TALE DNA-binding domains to at least 25 different TALE DNA-binding domains, 8 different TALE DNA-binding domains to at least 20 different TALE DNA-binding domains, at least 8 different TALE DNA-binding domains to at least 16 different TALE DNA-binding domains, or 8 different TALE DNA-binding domains to at least 12 different TALE DNA-binding domains.

c. Zinc Finger Nuclease (ZFN) System

The gene editing system of the present disclosure may include a ZFN-based gene editing system. The ZFN-based gene editing system may include a zinc finger DNA-binding domain and a nuclease, or DNA encoding the nuclease and the zinc finger DNA-binding domain, or mRNA for synthesis of the nuclease and zinc finger DNA-binding domain. In some embodiments, the water-in-oil droplets may comprise from about 0.1 μM to about 150 UM of a zinc finger DNA-binding domain and from about 0.1 μM to about 150 UM of a nuclease. In other embodiments, the water-in-oil droplets may comprise from about 1 μg to about 1 μg of DNA encoding the ZFN-based gene editing system. The concentration of the ZFN-based gene editing systems and buffers for supporting delivery of the ZFN-based gene editing systems are well established and known in the art.

A zinc finger protein is a protein that includes one or more zinc finger domains. Zinc finger domains are relatively small protein motifs that contain multiple finger-like protrusions that make tandem contacts with their target molecule such as a DNA target molecule. A zinc finger domain may bind one or more zinc ions or other metal ions such as iron, or in some cases a zinc finger domain forms salt bridges to stabilize the finger-like folds. The zinc binding portion of a zinc finger protein may include one or more cysteine residues and/or one or more histidine residues to coordinate the zinc or other metal ion. A zinc finger protein recognizes and binds to a particular DNA sequence via the zinc finger domain. In some embodiments, a zinc finger protein is fused to or includes a nuclease domain and may be referred to as a zinc finger nuclease (ZFN). The nuclease domain may include, for example, the endonuclease Fokl. ZFNs may recognize target sites that consist of two zinc-finger binding sites that flank a 5- to 7-base pair (bp) spacer sequence recognized by the endonuclease Fokl cleavage domain.

In some embodiments, the number of zinc finger DNA-binding domains that may be included in the ZFN-based gene editing system can be at least 1 zinc finger DNA-binding domain, at least 2 different zinc finger DNA-binding domains, at least 3 different zinc finger DNA-binding domains, at least 4 different zinc finger DNA-binding domains, at least 5 different zinc finger DNA-binding domains, at least 6 different zinc finger DNA-binding domains, at least 7 different zinc finger DNA-binding domains, at least 8 different zinc finger DNA-binding domains, at least 9 different zinc finger DNA-binding domains, at least 10 different zinc finger DNA-binding domains, at least 11 different zinc finger DNA-binding domains, at least 12 different zinc finger DNA-binding domains, at least 13 different zinc finger DNA-binding domains, at least 14 different zinc finger DNA-binding domains, or at least 15 different zinc finger DNA-binding domains. The number of zinc finger DNA-binding domain molecules that may be included in the ZFN-based gene editing system can be less than 30 different zinc finger DNA-binding domains, less than 25 different zinc finger DNA-binding domains, less than 20 different zinc finger DNA-binding domains, less than 19 different zinc finger DNA-binding domains, less than 18 different zinc finger DNA-binding domains, less than 17 different zinc finger DNA-binding domains, less than 16 different zinc finger DNA-binding domains, less than 15 different zinc finger DNA-binding domains, less than 14 different zinc finger DNA-binding domains, less than 13 different zinc finger DNA-binding domains, less than 12 different zinc finger DNA-binding domains, less than 11 different zinc finger DNA-binding domains, less than 10 different zinc finger DNA-binding domains, less than 9 different zinc finger DNA-binding domains, less than 8 different zinc finger DNA-binding domains, less than 7 different zinc finger DNA-binding domains, less than 6 different zinc finger DNA-binding domains, less than 5 different zinc finger DNA-binding domains, less than 4 different zinc finger DNA-binding domains, less than 3 different zinc finger DNA-binding domains, or less than 2 different zinc finger DNA-binding domains. The number of zinc finger DNA-binding domains that may be included in the ZFN-based gene editing system can be between at least 1 zinc finger DNA-binding domain to at least 30 different zinc finger DNA-binding domains, at least 1 zinc finger DNA-binding domain to at least 25 different zinc finger DNA-binding domains, at least 1 zinc finger DNA-binding domain to at least 20 different zinc finger DNA-binding domains, at least 1 zinc finger DNA-binding domain to at least 16 different zinc finger DNA-binding domains, at least 1 zinc finger DNA-binding domain to at least 12 different zinc finger DNA-binding domains, at least 1 zinc finger DNA-binding domain to at least 8 different zinc finger DNA-binding domains, at least 1 zinc finger DNA-binding domain to at least 4 different zinc finger DNA-binding domains, at least 4 different zinc finger DNA-binding domains to at least 30 different zinc finger DNA-binding domains, at least 4 different zinc finger DNA-binding domains to at least 25 different zinc finger DNA-binding domains, at least 4 different zinc finger DNA-binding domains to at least 20 different zinc finger DNA-binding domains, at least 4 different zinc finger DNA-binding domains to at least 16 different zinc finger DNA-binding domains, at least 4 different zinc finger DNA-binding domains to at least 12 different zinc finger DNA-binding domains, at least 4 different zinc finger DNA-binding domains to at least 8 different zinc finger DNA-binding domains, 8 different zinc finger DNA-binding domains to at least 30 different zinc finger DNA-binding domains, at least 8 different zinc finger DNA-binding domains to at least 25 different zinc finger DNA-binding domains, 8 different zinc finger DNA-binding domains to at least 20 different zinc finger DNA-binding domains, at least 8 different zinc finger DNA-binding domains to at least 16 different zinc finger DNA-binding domains, or 8 different zinc finger DNA-binding domains to at least 12 different zinc finger DNA-binding domains.

d. DNA-Binding Fusion Protein

Additionally or alternatively, a zinc finger protein or TALE can be fused to a polypeptide domain and referred to as a “DNA-binding fusion protein”. The DNA-binding fusion protein may act as a synthetic transcription factor. A zinc finger protein or TALE can be fused to a polypeptide domain having epigenetic modifying activity to mediate targeted gene regulation. For example, the DNA-binding fusion protein may include a polypeptide domain having transcription repression activity. A DNA-binding fusion protein comprising a zinc finger protein or TALE, and a polypeptide domain having transcription repression activity may mediate targeted gene repression. The polypeptide domain having transcription repression activity may comprise Kruppel associated box activity such as a KRAB domain or KRAB, MECP2, ERF repressor domain (ERD), Mad mSIN3 interaction domain (SID) or Mad-SID repressor domain, SID4× repressor domain, Mxil repressor domain, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof.

In other embodiments, the DNA-binding fusion protein includes a polypeptide domain having nuclease activity. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease. In some embodiments, the polypeptide domain having nuclease activity comprises Fokl.

### 4. BARCODE

Provided herein are barcode systems that may comprise one or more barcode polynucleotides or oligonucleotides. The term “barcode” or “barcode polynucleotide” or “barcode oligonucleotide” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. The barcode sequence may provide a high-quality individual read of a barcode associated with a subject, a single cell, a vector, labeling ligand (e.g., an aptamer), protein, shRNA, sgRNA, or cDNA such that multiple species can be sequenced together. Barcode technologies are known in the art and are described in Winzeler et al. (1999) Science 285:901; Brenner (2000) Genome Biol. 1:1; Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240. Barcodes may be single-stranded or double-stranded.

The barcodes may comprise one or more primer sequences. The one or more primer sequences may be at the 5′ and/or 3′ ends of the barcode polynucleotides. The primer sequences may be a promoter sequence known in the art, a terminator sequence known in the art, or a combination thereof. For example, the promoter sequence may be a T7 promoter or a SP6 promoter, and the terminator sequence may be a T7 terminator. The barcodes may comprise one or more spacer sequences. The barcodes may be unmodified. The barcodes may comprise an end-cap modification at the 5′ end of the barcode. The end-cap modification may be any modification that prevents exonuclease and/or endonuclease degradation of the barcode. For example, the end-cap medication may be biotinylation, 2′OMe, phosphorothioate, or a combination thereof. In an embodiment, the barcode may be double-stranded DNA and comprise biotin at the 5′ end on both the sense and antisense strands. In another embodiment, the barcode may be mRNA or gRNA. In another embodiment, the barcodes may be genome integrateable ssoligo or dsDNA with homology arms for targeted insertion. In another embodiment, the barcodes may be attached to a solid support such as polymer beads. In another embodiment, the barcodes may be optical barcodes such as microbeads loaded with quantum dots/nanospheres (Hu et al. (2018) Nat Methods 15, 194-200; Han et al. (2001) Nat Biotechnol. 19, 631-635). In another embodiment, the barcodes may be spatially organizing fluorescent molecules such as Nanostrings (Geiss et al. (2008) Nat Biotechnol. 26, 317-325) or fluorescently-labeled DNA nanorods (Lin et al. (2012) Nature Chem. 4, 832-839).

A barcode may be may comprise a oligonucleotide or polynucleotide sequence of at least about 5 nt or bp, at least about 10 nt or bp, at least about 15 nt or bp, at least about 20 nt or bp, at least about 25 nt or bp, at least about 30 nt or bp, at least about 35 nt or bp, at least about 40 nt or bp, at least about 45 nt or bp, at least about 50 nt or bp, at least about 55 nt or bp, at least about 60 nt or bp, at least about 65 nt or bp, at least about 70 nt or bp, at least about 75 nt or bp, at least about 80 nt or bp, at least about 85 nt or bp, at least about 90 nt or bp, at least about 95 nt or bp, at least about 100 nt or bp, at least about 105 nt or bp, at least about 110 nt or bp, at least about 115 nt or bp, at least about 120 nt or bp, at least about 125 nt or bp, at least about 130 nt or bp, at least about 135 nt or bp, at least about 140 nt or bp, at least about 145 nt or bp, or at least about 150 nt or bp in length, that is specific for a DNA fragment. A barcode may be may comprise a oligonucleotide or polynucleotide sequence of less than about 150 nt or bp, less than about 145 nt or bp, less than about 140 nt or bp, less than about 135 nt or bp, less than about 130 nt or bp, less than about 125 nt or bp, less than about 120 nt or bp, less than about 115 nt or bp, less than about 110 nt or bp, less than about 105 nt or bp, less than about 100 nt or bp, less than about 95 nt or bp, less than about 90 nt or bp, less than about 85 nt or bp, less than about 80 nt or bp, less than about 75 nt or bp, less than about 70 nt or bp, less than about 65 nt or bp, less than about 60 nt or bp, less than about 55 nt or bp, less than about 50 nt or bp, less than about 45 nt or bp, less than about 40 nt or bp, less than about 35 nt or bp, less than about 30 nt or bp, less than about 25 nt or bp, less than about 20 nt or bp, less than about 15 nt or bp, or less than about 10 nt or bp in length, that is specific for a DNA fragment. A barcode may be specific for one DNA fragment. For example, a sequence for a gene made up of multiple DNA fragments may be associated with multiple barcodes.

In some embodiments, the water-in-oil droplets may comprise from about 1 ng/μL to about 100 ng/μL, about 1 ng/μL to about 50 ng/μL, about 1 ng/μL to about 40 ng/μL, about 1 ng/μL to about 30 ng/μL, about 1 ng/μL to about 20 ng/μL, or about 1 ng/μL to about 10 ng/μL of one or more DNA barcode(s). The concentration of the barcode systems and buffers for supporting delivery of the barcode systems are well established and known in the art. The one or more barcodes may be generated using any sequence, including sequences unrelated to the target gene. The one or more barcodes may be generated using one or more templates used for generation of a gene editing system as described herein. For example, a barcode may be generated using a DNA template used for generation of a gRNA molecule. Another example provides a barcode that may be generated using a DNA template used for generation of a TALE DNA-binding domain. Another example provides a barcode that may be generated using a DNA template used for generation of a zinc finger DNA-binding domain.

### 5. ADMINISTRATION

The droplets as detailed herein, or at least one component thereof, may be administered or delivered to a subject. Such droplets can comprise gene editing systems and barcodes in dosages well known to those skilled in the art taking into consideration such factors as the age, sex, weight, and condition of the particular subject, and the route of administration. The droplets as detailed herein, or at least one component thereof, may be administered to a subject by injection such as microinjection. The droplets as detailed herein, or at least one component thereof, may be administered by, for example, traditional syringes, micropipettes, microinjectors, electroporation, orally such as by feeding droplets to a subject, or needleless injection devices. In an embodiment, the droplets as detailed herein, or at least one component thereof, may be administered to an embryo.

Upon delivery of the presently disclosed droplets, or at least one component thereof, and thereupon a gene editing system and barcode(s) into the cells of the subject, the cells may express a gene editing system as described herein.

### 6. METHODS

a. Methods for Large-Scale Identification of a Gene In Vivo

Provided herein are methods for large-scale identification of a gene in vivo in a plurality of subjects. The methods may include administering to a plurality of subjects a plurality of the barcode polynucleotides or oligonucleotides described herein by methods described herein, isolating one or more of the barcode polynucleotides or oligonucleotides from the plurality of subjects, amplifying the isolated barcode polynucleotides or oligonucleotides, and sequencing the amplified barcode polynucleotides or oligonucleotides.

Isolating may comprise selecting one or more subjects from the plurality of subjects that exhibit one or more phenotypes of interest. For example, a phenotype of interest may be a behavioral phenotype such as movement or morphological phenotype such as craniofacial defects. Isolating may further comprise lysing the plurality of subjects that exhibit one or more phenotypes of interest or cells therefrom, removing excess unbound barcodes from the plurality of subjects by, for example, washing, and amplifying the barcodes. Amplifying the isolated barcodes may comprise mixing the barcodes with one or more primers such as a primer set. At least a portion of the primers may anneal to the 5′ and 3′ ends of the barcode thereby allowing for use of many different amplification primers, but one sequencing primer. This allows for more consistent sequencing results than if a gene-specific primer was used as both the amplification and sequencing primer. For example, a M13F and M13R sequence may be added to the barcodes during amplification and a M13F or M13R primer may be used for sequencing of all the barcodes that comprise the M13F and M13R sequences. The barcodes may be amplified with the primers using PCR amplification and a polymerase such as Taq polymerase using protocols that are well known in the art. The amplified barcode products may be enzymatically cleaned using, for example, one or more exonucleases known in the art and one or more phosphatases known in the art.

Sequencing the amplified barcodes can be performed using variety of sequencing methods known in the art including, but not limited to, sequencing by hybridization (SBH), sequencing by ligation (SBL), Sanger sequencing, quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., on cyclic array sequencing using platforms such as Roche 454, Illumina Solexa, ABI-SOLID, ION Torrents, Complete Genomics, Pacific Bioscience, Helicos, Polonator platforms (Worldwide Web Site: Polonator.org), and the like, can also be utilized. High-throughput sequencing methods are described in U.S. Pat. Pub. No. 2010/0273164. A variety of light-based sequencing technologies are known in the art (Landegren et al. (1998) Genome Res. 8:769-76; Kwok (2000) Pharmocogenomics 1:95-100; and Shi (2001) Clin. Chem. 47:164-172).

b. Methods for Large-Scale Identification of Gene Function

Provided herein are methods for large-scale identification of a gene function in a plurality of subjects. The methods may include administering to a plurality of subjects a plurality of the droplets comprising a gene editing system and one or more barcodes as detailed herein, or at least one component thereof as described herein; isolating the one or more barcode polynucleotides or oligonucleotides from the plurality of subjects as detailed herein; amplifying the isolated one or more barcode polynucleotides or oligonudeotides as detailed herein; and, sequencing the amplified one or more barcode polynucleotides or oligonucleotides as described herein. The method may also comprise selecting the plurality of subjects with one or more phenotypes of interest before isolating the one or more barcodes as described herein. Each subject of the plurality of subjects may be administered one droplet comprising a gene editing system that targets a different gene in each subject. The plurality of droplets may be administered to the plurality of subjects simultaneously. The water-in-oil droplets may be used to target multiple different genes simultaneously by delivering multiple water-in-oil droplets that each comprise a gene editing system that targets a different gene to multiple organisms concurrently.

The method may also include identifying differentially expressed genes in the plurality of subjects, in particular in an organ of interest before designing the gene editing system and administering the plurality of droplets. The differentially expressed genes may be enriched by removing duplicates and unannotated genes. The enriched genes may be further enriched for poorly characterized genes by removing genes with known phenotypes. Then, the gene editing system may be designed to target the poorly characterized genes to correlate the genes with a phenotype.

### 7. KITS

Provided herein is a kit, which may be used to identify a gene in vivo in a plurality of subjects. The kit may comprise barcodes or a composition comprising the same, for identification of a gene in vivo, as described above, and instructions for using said barcodes or composition. In an embodiment, the kit comprises at least one barcode and instructions for using the barcode.

Also provided herein is a kit, which may be used to identify a gene function in a plurality of subjects. The kit may comprise droplets or a composition comprising the same, for identification of a gene function, as described above, and instructions for using said droplets or composition. In an embodiment, the kit comprises at least one droplet system that comprises at least one gene editing system, at least one barcode, at least one fluorinated oil, and at least one fluorosurfactant, and instructions for using and/or making the droplet system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 8. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Zebrafish husbandry and breeding. All protocols related to zebrafish (Danio rerio) were approved by the Institutional Animal Care and Use Committee at the University of Utah (Protocol #19-09011). Adult TuAB strain zebrafish and Tg(cmlc2:NdsRed) were maintained in the Centralized Zebrafish Animal Resource (CZAR) core at 28-29° C. with a 14/10 light/dark cycle. Tg(cmlc2:eGFP) zebrafish were maintained in HJY lab (Eccles Institute of Human Genetics). To produce embryos, adult zebrafish in a 1:1 male:female ratio were placed in a breeding tank and separated by a divider overnight. Embryos were collected after removing the divider in the morning.

Guide RNA (gRNA) design and selection criteria. All gRNAs were designed using CHOPCHOP version 3.0.0 (chopchop.cbu.uib.no). The targets were specified using the Gene ID or the ENSEMBL ID. “danRer10/GRCz10” was used as the reference sequence. The single gRNAs (sgRNAs) were designed for “knock-out” using “CRISPR/Cas9” from Streptococcus pyogenes with “NGG” as the PAM sequence. The sgRNA length without PAM was specified as “20” except in certain circumstances (see below) when “19” bases length was used. The default methods for determining off-targets in the genome “Off-targets with up to 3 mismatched in protospacer (Hsu et al. (2013) Nat Biotechnol 31, 827-832)”; and an efficiency score calculation based on “Doench et al. (2016) Nat Biotechnol 34, 184-191-only for NGG PAM” were used. The 5′ requirement for sgRNA was changed to “GN or NG” and the software used Thyme et al. (2016) Nat. Commun. 7:11750 to “Check for self-complementarity” and to “Check for self-complementarity versus a Standard backbone (AGGCTAGTCCGT)”. All other functions were kept at default options. The following criteria was followed to select 4 targets per gene: (1) Targets of 20 bp length in the early to middle exons that start with “GA” and had no off-targets with fewer than 3 bp mismatches were prioritized. (2) If guides that met criterion 1 could not be found, guides that started with “GA” and were 19 bp in length were used. (3) If criterion 1 and 2 were not met, gRNAs that started with “GN” were picked. If it was not possible to design gRNA with no off-targets, guides with at least 3-bp mismatches of which at least 1 mismatch was in seed region were selected. All gRNAs had 45-80% GC content. The gRNA sequences are listed in TABLE 1 and Supplementary Table 5 of Parvez et al. (2021) Science. 373:6559, 1146-1151, which is incorporated herein by reference in its entirety. No unique gRNAs could be designed for six of the candidate genes.

TABLE 1
gRNA spacer sequences targeting chrd, fgf24, npas4l, rx3, tbx5a, tbx16, tnnt2a,
trpa1b, and tyr.

| | Sequence | | SEQ ID |
|---|---|---|---|
| Gene name | number | Spacer Sequence | NO: |
| tyrosinase | tyr-1 | GAAAGTTACAACCTCCGCG | 13 |
| | tyr-2 | GATGTTGGCGAACATTGGCG | 14 |
| | tyr-3 | GAACCTCTGCCTCTCGGTAG | 15 |
| | tyr-4 | GATACTGCGGCCCGTTGGGA | 16 |
| troponin T type 2a | tnnt2a-1 | GACATCCACCGTAAGCGCA | 17 |
| | tnnt2a-2 | GAAGAGACCACTCAGGAACA | 18 |
| | tnnt2a-3 | GCGCTTACGGTGGATGTCCT | 19 |
| | tnnt2a-4 | GCTCCCTTTCGCGTTCGCTG | 20 |
| T-box transcription factor 5a | tbx5a-1 | GACGTGACCGCAATGAACG | 21 |
| | tbx5a-2 | GTATGTAGTCTGCGATGACG | 22 |
| | tbx5a-3 | GTCTTCACTGTCCGCCATGT | 23 |
| | tbx5a-4 | GGAGTTCAAGATGATCTGCG | 24 |
| T-box transcription factor 16 | tbx16-1 | GAAGCTCACCAATAACGCAC | 25 |
| | tbx16-2 | GTACGTCCTGTAGGGCGGCT | 26 |
| | tbx16-3 | GGAATCACCGGCTCCGGGCA | 27 |
| | tbx16-4 | GTGGACATGGTACCAGAAGA | 28 |
| fibroblast growth factor 24 | fgf24-1 | GACGACGTGAGCCGAAAGC | 29 |
| | fgf24-2 | GATGGGGGCAAGTACGGTA | 30 |
| | fgf24-3 | GGCTCACGTCGTCTCGAGTG | 31 |
| | fgf24-4 | GGCAAACACGTGCAAATTCT | 32 |
| chordin | chrd-1 | GAGCTCCAGTGGTGTCGCGA | 33 |
| | chrd-2 | GACGGGTGTGACAGACTCT | 34 |
| | chrd-3 | GATCGTCGCAGGTCGGATC | 35 |
| | chrd-4 | GACACGTGGCATCCAGATCT | 36 |
| neuronal PAS domain protein | npas4l-1 | GTAAAGGCAACGATAAACCC | 37 |
| 4 like | npas4l-2 | GACGGATCCGCACCAGCAGG | 38 |
| | npas4l-3 | GATTGCGGCGTGGCGGTCAG | 39 |
| | npas4l-4 | GTTCCACCTGGGCTTCTCAG | 40 |
| | npas4l-5 | GAGAACGTACACGAGTATC | 41 |
| retinal homeobox gene 3 | rx3-1 | GATCTGCCAGACGCGGATGG | 42 |
| | rx3-2 | GAGCTCGTGGAGCTGGAAGG | 43 |
| | rx3-3 | GGGAGAGACTCTGTTTCACC | 44 |
| | rx3-4 | GAGCACTTGTCCCCGAAAA | 45 |
| | rx3-5 | GAACGTGGTTCGGTTCCGC | 46 |
| transient receptor potential | trpa1b-1 | GATATCGTCAACATTCGGGA | 47 |
| cation channel, subfamily A, | trpa1b-2 | GGCACCGCGCTTGATCTGTA | 48 |
| member 1b | trpa1b-3 | GCGAAAGCAACAGTATGAAT | 49 |
| | trpa1b-4 | GTACGCGGAGGCAATATCG | 50 |
| scrambled (non-targeting) | scr-1 | GATTAGTCGGTGCGCGTGAA | 51 |
| | scr-2 | GGAGCATGTACGAGTTGCTG | 52 |
| | scr-3 | GATCCGCCTGTAGTCTCGCA | 53 |
| | scr-4 | GACGGGCAGTCTAGCGTGTC | 54 |

In vitro transcription. The DNA templates for in vitro transcription (IVT) were generated using fill in PCR of a target-specific forward oligo and a constant reverse oligo as reported in Gagnon et al. (2014) PLoS ONE 9(5): e98186. Target-specific forward oligos ATTTAGGTGACACTATA(N)19/20GTTTTAGAGCTAGAAATAGCAAG (SEQ ID NO: 59) containing a SP6 RNA polymerase site followed by 19 or 20 bp of the gRNA sequences were ordered from IDT as 25 nmol desalted and lyophilized powder. The constant reverse oligo AAAAGCACCGACTCGGTGCCACTTTTTCAAGTTGATAACGGACTAGCCTTATTTTAACTTGC TATTTCTAGCTCTAAAAC (SEQ ID NO: 60) was synthesized at the University of Utah DNA synthesis core and HPLC purified. Both the forward and reverse oligos were dissolved in nuclease free H₂O (Invitrogen; cat #AM9906) to a 100 UM concentration. Oligos forthe screen were ordered in 96-well plate as 500 pmol desalted and lyophilized powder and reconstituted in water to a concentration of 10 μM. To generate the double stranded DNA template, a reaction mix containing 1×HF buffer (NEB; cat #B0518S), 1 μM each of forward oligo and the constant reverse oligo, 200 UM dNTPs (Fisher Scientific; cat #R0194), 3% DMSO (v/v), and 1 U of Phusion HS Flex DNA polymerase (NEB, cat #M0535L) was made. The PCR mix was placed in a thermal cycler (Bio-Rad) and incubated at 98° C. for 2 min, 50° C. for 10 min, 72° C. for 10 min, after which the temperature was reduce to 4° C. The sample was cleaned up using a Zymo DNA Clean and Concentrator®-5 kit (Zymo Research, cat #D4013). For larger number of samples, a ZR96 DNA Clean and Concentrator®-5 clean up kit was used (Zymo Research, cat #D4024). The double stranded DNA was eluted in 15 μL nuclease free water, concentration determined using a Nanodrop™ (Thermo Scientific), DNA integrity assessed using DNA gel electrophoresis, and then stored at −20° C. IVT was performed in RNAse free condition using a MEGAscript™ SP6 Transcription kit (Thermo Fisher Scientific, cat #AM1330) according to manufacturer's guidelines. For each reaction of 20 μL, 6 pmol of total multiplexed DNA (4×1.5 pmol each DNA) as well as 0.25 μL of RNAse inhibitor (Thermo Fisher Scientific; cat #EO0382) was used. The IVT sample was incubated at 37° C. overnight (˜16 h), after which the sample was treated with 1 μL Turbo™ DNAse for 15 min at 37° C. Subsequently, the samples were cleaned up using an RNA Clean and Concentrator®-5 (Zymo Research, cat #R1013) or a ZR96 RNA Clean and Concentrator®-5 (Zymo Research, cat #R1080) and eluted in 12 μL nuclease free water. The RNA concentration was determined using a Nanodrop™ (Thermo Scientific), RNA integrity assessed using gel electrophoresis, and the samples were then stored at −80° C.

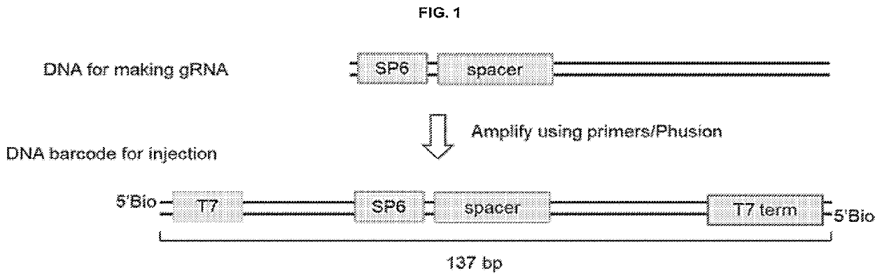

Barcode Generation. The DNA barcodes were generated by extending and putting a 5′-Biotin group on the DNA template used for IVT (FIG. 1). Any one of the four DNA templates used for gRNA generation was used for barcode generation. A set of forward primer/5BiosG/CGTAATACGACTCACTATAGGGCTTCAGCCAAGGAAGCTACATTTAGGTGCACTAA G (IDT; SEQ ID NO: 55) and reverse primer/5BiosG/GCTAGTTATTGCTCAGCGGGTCTTGTTTCTCGGTGTGCTTGCTATTTCTAGCTCTA AAAC (IDT; SEQ ID NO: 56) was used to amplify the barcode using Phusion® HS Flex DNA polymerase following standard protocol. The 5′-Biotin was added to enable enrichment of the barcode for more efficient recovery.

Droplet generation. The CRISPR droplets were generated using a QX200 Droplet generator (Bio-Rad, cat #1864002) using 3%008-Surfactant (w/v) (Ran Biotechnologies; cat #008-FluoroSurfactant-1G) in Novec™-7500 oil (Gallade Chemical, cat #HFE-7500) (3% HFE for here on). Several oils and surfactants and combinations thereof were tested for toxicity, stability, and consistency of injection (TABLE 2; the more +s, the better the result). First, a mix containing 5000 ng of total gRNAs (4 gRNA/genes), 4.2 μL of 20 UM EnGen® Cas9 (NEB, cat #M0646M), 2.5 UL of 10× Buffer 3.1 was made in nuclease free water and incubated at room temperature for 10 min. Subsequently, 250 ng of DNA barcode and 3.5 μL of 0.5% Phenol Red dye in PBS (Sigma, cat #P0290) was added to the mix. The final volume of the RNP mix was 25 μL with final concentrations of 200 ng/μL gRNAs, 3.36 UM EnGen® Cas9 nuclease, 1× Buffer 3.1, 10 ng/μL DNA barcode, and 0.07% of Phenol Red. The sample was gently mixed and 20 μL of it was transferred to the cartridge (Bio-Rad, cat #1864007) using a 20 μL multichannel pipet (Rainin). QX200™ can generate droplets for 8 samples per cartridge. If preparing droplets for less than 8 samples, the remaining wells were filled with 20 μL sample containing 1× Droplet generation buffer (Bio-Rad, cat #1863052). 3% HFE was then loaded in the designated wells in the cartridge. The cartridge was loaded on the cartridge holder (Bio-Rad) sealed using a rubber gasket (Bio-Rad, cat #1864007) and placed in the QX200™ Droplet generator. Once droplet generation was complete (˜2 min/8 samples), the droplets were immediately transferred to PCR strip tubes (Fisher Scientific) containing 50 μL 3% HFE using a 200 μL multichannel pipet (Rainin). The droplets float on the oil surface because of higher density of the oil than the aqueous droplets. The droplets were used immediately or stored at 4° C. for up to a month in capped PCR strip tubes. If intermixing droplets from different samples, 2 μL droplets from each sample was combined into a separate PCR tube containing 3% HFE. For our screen, we intermixed droplets from 50 different samples. The samples were mixed gently for even distribution. Care was taken during droplet transfer and mixing to avoid droplet fusion. P-20 and P-200 tips, because of their wider tip width, were used for transfer and mixing, respectively.

TABLE 2
Effects of oil and surfactant combinations on toxicity, stability, and
consistency of injection.

| Oil + surfactant | Non-Toxic to | Stable for | Consistent |
|---|---|---|---|
| tested | embryos? | storage? | injection? |
| Bio-Rad Droplet | Not tested | + | Not tested |
| Generation Oil for | | | |
| EvaGreen ® | | | |
| Bio-Rad Droplet | ++ | +++ | ++ |
| Generation Oil for | | | |
| Probes | | | |
| 2% (wt/v) 008- | +++ | +++ | ++ |
| fluorosurfactant in | | | |
| HFE-7500 | | | |
| 3% (wt/v) 008- | +++ | +++ | +++ |
| fluorosurfactant | | | |
| in HFE-7500 | | | |
| 5% (wt/v) 008- | ++ | +++ | ++ |
| fluorosurfactant in | | | |
| HFE-7500 | | | |

Droplet injection. All injections were performed in embryos at the 1-cell stage using a Microinjection system Pico-injector (Harvard Apparatus) fitted with a dissecting microscope (Leica Microsystems). The needles (Sutter Instrument, cat #TW100F-3) for microinjection were pulled using a P-1000 Micropipette puller (Sutter Instrument) at the following setting: Heat: 565, Pull: 64, Velocity: 77, Time: 80, and Pressure: 500. Around 300-500 droplets were transferred (along with the 3% HFE carrier oil) into a microinjection needle using a Microloader™ tip (Eppendorf; cat #5242956.003). 3 μL volume setting on a P-20 μL pipette typically transfers 300-500 droplets. The needle was gently flicked to get rid of any trapped air bubble. Care was taken to avoid vigorous shaking during transfer or flicking. The injection needle was attached to the injector and trimmed such that the opening width was around 10-20 microns. Because of the density difference between the oil and the aqueous droplets, the droplets collect at the top in the injection needle. The “Clear” setting was used to gently push out the excess 3% HFE carrier oil before injection. Once the droplets move near the tip, the injection can proceed. Embryos were placed in an injection mold. After injecting one droplet, the oil between two consecutive droplets was injected out in the mold, followed by injection of the subsequent droplet in the next embryo. 300-500 droplets were injected from a single injection needle in one morning. After injection, the embryos were transferred to a petri dish, washed once with E3 medium (5 mM NaCl, 0.17 mM KCl, 0.33 mM CaCl₂), 0.33 mM MgSO₄) to get rid of any carrier oil and residual RNP mix, split into multiple dishes (50-60 embryos per dish) to avoid overcrowding, and raised at 28.5° C. in E3 medium with methylene blue.

Phenotype screening. 24 hours post injection embryos were screened for any morphological phenotypes using a SteREO Discovery. V8 dissecting microscope (Zeiss). Dead embryos were removed, and the old media was replaced with fresh E3 media. Embryos showing gross morphological defects caused by general nucleic acid toxicity (˜15%) were also removed. The embryos were screened at multiple different time points-24 hours post fertilization (hpf), 30 hpf, 48 hpf, 72 hpf—and any embryos showing cardiovascular phenotypes were isolated.

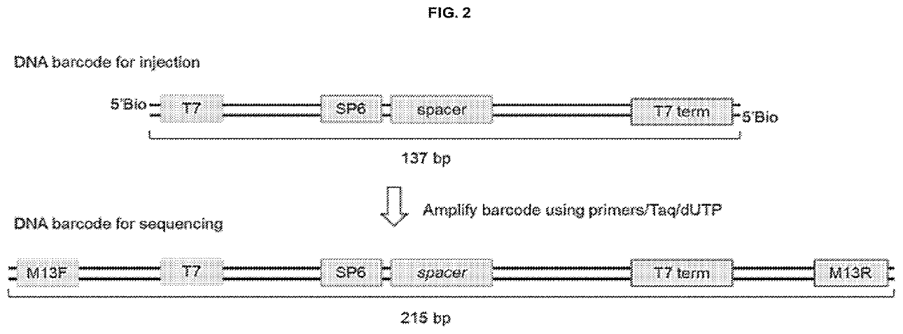

Barcode retrieval and sequencing. To identify the specific gene targeted by MIC-Drop CRISPR editing that was responsible for the phenotype-of-interest, the embryos showing the phenotype-of-interest were washed, transferred to a new plate and washed again 3× in E3 media to get rid of any residual DNA barcodes sticking to embryos. The embryos were then transferred to 10 μL of a 2× lysis buffer (20 mM Tris (pH 8), 4 mM EDTA, 0.4% Triton™ X-100) with freshly added Proteinase K (Sigma, cat #3115828001) at a concentration of 0.2 mg/mL. The 20 μL sample was incubated overnight at 50° C. for complete lysis. Proteinase K was heat inactivated the following morning by heating at 95° C. for 10 min. The lysate was mixed gently, centrifuged at 3000×g for 5 min to pellet the debris. The supernatant was collected and used for PCR amplification of the DNA barcode. A set of primers priming at the T7F (GTGTAAAACGACGGCCAGTATGGCACCAACTCGATGACGTAATACGACTCACTATAGGGC; SEQ ID NO: 57) and T7term (CAGGAAACAGCTATGACATAGTCCTGCTGTACCAGGCGTCTGCTAGTTATTGCTCAGCGG; SEQ ID NO: 58) were used to amplify the barcode. The barcode was amplified using Taq ploymerase (Promega, cat #M3008) using standard protocol. To prevent carryover contamination of barcodes, UDG (NEB, cat #M0280S) at a final concentration of 25 U/mL and 200 μM dNTPs (70:30 of dTTP:dUTP) was used in the PCR reaction. The amplified product was enzymatically cleaned using Exonuclease I (NEB, M0293) and shrimp alkaline phosphatase (NEB #M0371) using manufacturer's protocol. The barcode was sequenced using M13F or M13R primers. See FIG. 2.

Validation of editing efficiency. Editing efficiency was analyzed using either a T7 endonuclease (T7E1) assay or Amplicon sequencing. For T7E1 assay, the targeted region was amplified using Q5 high fidelity polymerase (NEB, cat #M0493S) and a set of primers flanking the cut site. 200 ng of the cleaned amplified product was first denatured and then reannealed by gradual cooling according to the manufacturer's protocol. The sample was treated with 10 U of T7E1 enzyme (NEB, cat #M0302S) in a total volume of 20 UL and incubated at 37° C. for 15 min. EDTA at a final concentration of 25 mM was added to quench the reaction. The samples were resolved on a 2% agarose gel. For Amplicon sequencing, 150-500 bp amplicons from the targeted regions were sequenced on an Illumina platform using paired reading at a depth of 50,000 reads (Genewiz, Amplicon-EZ). Amplicon sequencing data were analyzed using Cas-Analyzer (rgenome.net/cas-analyzer/#!).

Light- and Optovin-induced motor response assay. Zebrafish larvae at 3 dpf were arrayed in 96-well plates and treated with 10 μM optovin (Fisher Scientific, cat #490110) in a total volume of 200 μL E3 media. Treated larvae were incubated at 37° C. for 1 h in dark. Subsequently, light-dependent motor response was assayed using a Zebrabox platform (ViewPoint Behavior Technology). Movement of the larvae was tracked and quantitated following 5×1s pulse of violet light after 10 s interval in the dark.

Computational pipeline to identify high-confidence genes for CRISPR screen. Raw RNA-seq data files (paired Fastq) were downloaded from the Gene Expression Omnibus (Accession #GSE85416) (Wang et al. (2017) Scientific Reports 7, 1250-1250; Shih et al. (2015) Circulation. Cardiovascular genetics 8, 261-269). Transcript abundances were quantified using kallisto and genome build GRCz10 release 89 (may2017.archive.ensembl.org) for all samples. Estimated counts for all transcripts per gene were summed to give a gene-level abundance estimation. Estimated counts were rounded to the nearest integer and subset to perform two separate differential expression analyses, the first comparing zebrafish larval heart samples (SRR4017367, SRR4017368, SRR4017369) to zebrafish adult heart samples (SRR4017370, SRR4017371, SRR4017372) and the second comparing the aforementioned adult samples to zebrafish adult muscle samples (SRR4017373, SRR4017374, SRR4017375). Genes with less than 10 counts across all samples (n=6803) were removed from the matrix prior to performing differential expression analysis. DESeq2 was run on each comparison using a negative binomial LRT model correcting for replicate (counts˜ replicate+tissue). To find genes that are in enriched in larval cardiac tissue, the data was filtered by fold change and by adjusted p-value (false discovery rate <1%). Genes that were significantly enriched in adult heart as compared to adult muscle (n=3488) and genes that were significantly enriched in larval heart as compared to adult heart (n=4150) were carried forward in the analyses. Out of these datasets, 465 genes were found to be overlapping in each filtered comparison. The gene list was manually curated to remove any genes that were already known to have cardiac phenotypes in various animal models or predicted gene models that have not been characterized/validated. The final gene list contained 188 genes found to be enriched in larval cardiac tissue without known phenotypes, and 6 control genes with expected outcomes.

Rescue assay. Codon-optimized gene sequences were ordered as gene fragments (Genewiz), amplified, and cloned in a pcs2+ vector using restriction enzymes. The gene sequences were amplified using RNA-fwd and RNA-Rev primers. mRNA was generated using a SP6 mMessage mMachine transcription kit (Thermo Fisher Scientific, cat #AM1340) per manufacturer's protocol. 1-1.5 nL of RNP containing 100 ng/μL gRNA, 2 μM Cas9, and 300 ng/μL mRNA was injected in embryos at 1-cell stage. Phenotype was analyzed at 3 dpf.

o-dianisidine staining. Zebrafish embryos at 3 dpf were stained in the dark for 30 min with a solution containing 0.6 mg/mL o-dianisidine, 0.01 M sodium acetate (pH 4.5), 0.65% H₂O₂, and 40% EtOH (v/v). Stained embryos were washed with water and then fixed in 4% paraformaldehyde (PFA) in phosphate-buffered saline (PBS) for 1 h. Next, embryos were treated for 30 min with a solution containing 0.8% KOH, 0.9% H₂O₂, and 0.1% Tween-20 to remove the pigments. Finally, the depigmented embryos were washed in 0.1% Tween-20 in PBS and then fixed with 4% PFA for at least 3 hours. All procedures were performed at room temperature. Embryos were stored in PBS at 4° C. and imaged using a Leica M205 FA Stereoscope.

Alcian blue stain. 5 dpf embryos were fixed in 4% PFA for 2 hours at room temperature. Embryos were dehydrated in 50% EtOH for 10 min at room temperature and then treated with a solution containing 0.04% alcian blue 8 GX (Sigma-Aldrich, cat #A5268), 0.005% alizarin red S (Sigma, cat #A5533), and 50 mM MgCl2 in 70% EtOH and incubated overnight with at 4° C. The embryos were washed with water once before depigmented using a solution containing 1% KOH and 1.5% H₂O₂ and treated for 20 min at room temperature. Next, tissues were cleared by washing with 0.25% KOH and 20% glycerol for 30 min at room temperature followed by another wash with 0.25% KOH and 50% glycerol. Samples were stored in 0.25% KOH and 50% glycerol at 4° C. and imaged using a Leica M205 FA Stereoscope.

Imaging. Tg(cmlc2:NdsRed) or Tg(cmlc2.eGFP) were euthanized by placing in 1% PFA for 5 min, embedded in agarose and imaged using a Zeiss LSM 700 confocal microscope. For live imaging, zebrafish larvae were anesthetized in 0.016% Tricaine in E3. Low magnification brightfield images were collected using a Leica M205 FA stereoscope. High magnification videos of zebrafish were collected using a Zeiss AXIO Observer. A1 microscope using a Metamorph software (Molecular Devices) at 10 fps. All images were processed and analyzed using ImageJ (NIH).

Voltage mapping. Optical mapping was performed as previously described (Panáková et al. (2010) Nature 466:7308 874-878). Briefly, hearts from 72 hpf zebrafish embryos were isolated in Tyrode's buffer and loaded with the transmembrane potential-sensitive dye, FluoVolt™ (Life Technologies, cat #F10488) for 20 min to measure the action potentials. After transferring the stained hearts to fresh Tyrode's buffer to remove excess dye, individual hearts were placed in chamber containing 0.05 mg/ml of the mechanical uncoupler Cytochalasin D (ThermoFisher Scientific, cat #PHZ1063) to inhibit contraction. Fluorescence intensities were recorded with an inverted microscope (TE-2000, Nikon) equipped with a high-speed CCD camera (RedShirtImaging) at a maximum frame rate of 2000 Hz. Propagation velocities and depolarization waves were extracted using custom scripts in MATLAB 9.5 software (Mathworks, version R2018b) as previously described (Panáková et al. (2010) Nature 466:7308 874-878). Briefly, activation times were defined as the time for 80% depolarization and isochronal maps representing the wavefront at fixed time intervals (10 ms) were calculated from the activation data using the contour-plotting function in MATLAB. Local conduction velocities of regions-of-interest (40 mm2 in size) were defined as previously described (Panáková et al. (2010) Nature 466:7308 874-878).

### Example 2

### Delivery and Analysis of Multiplexed Intermixed CRISPR Droplets

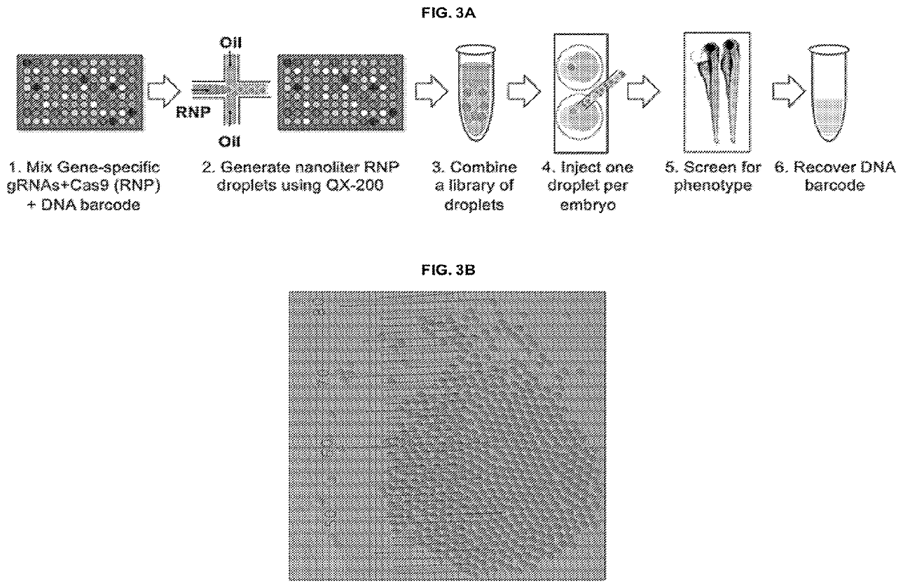

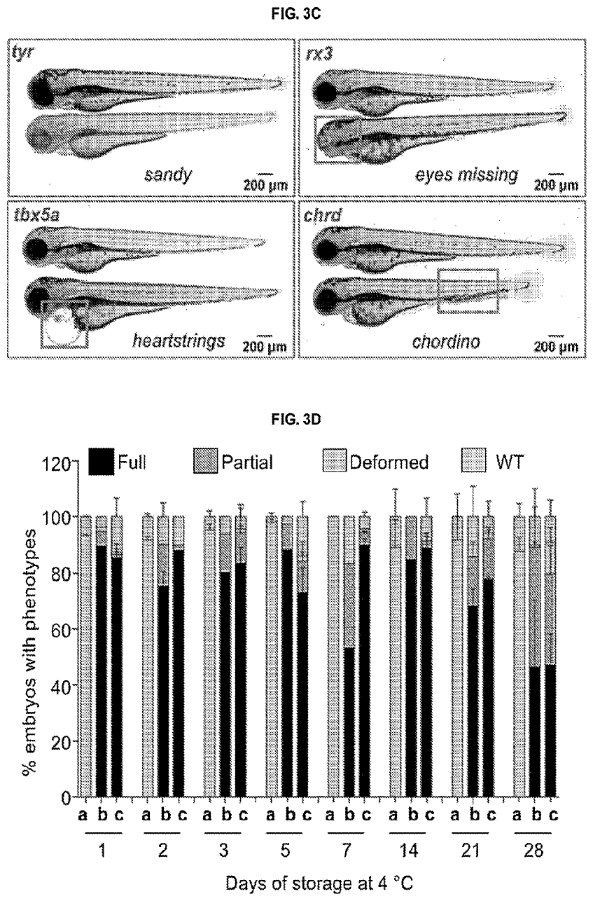

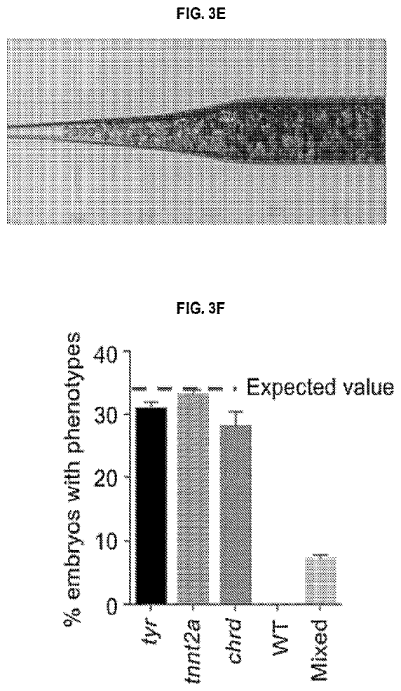

Described herein is a novel platform, Multiplexed Intermixed CRISPR Droplets (MIC-Drop), for performing large-scale reverse-genetic screens in zebrafish (FIG. 3A). The platform uses microfluidics to generate nanoliter-sized droplets, each droplet containing Cas9, multiplexed gRNAs targeting individual genes-of-interest, and a unique barcode associated with each target gene. Droplets targeting hundreds to thousands of different genes are intermixed together and injected into zebrafish embryos from a single needle. Embryos are raised en masse, those exhibiting phenotype(s)-of-interest are isolated, and the identities of the perturbed genes are rapidly uncovered by retrieving and sequencing the barcodes.

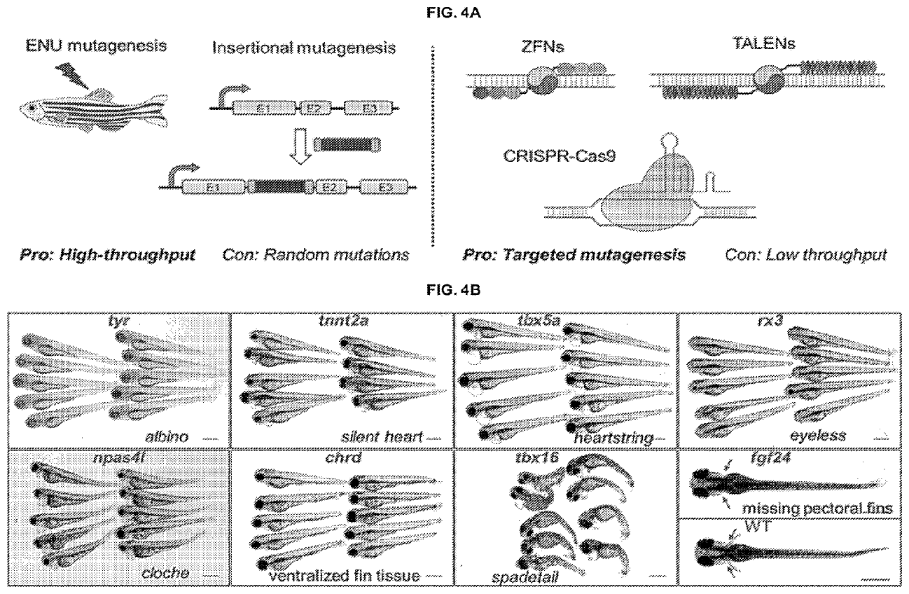

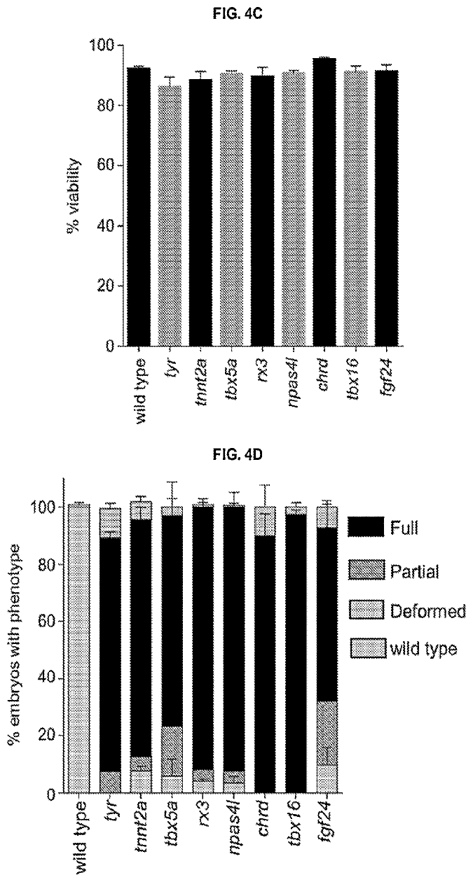

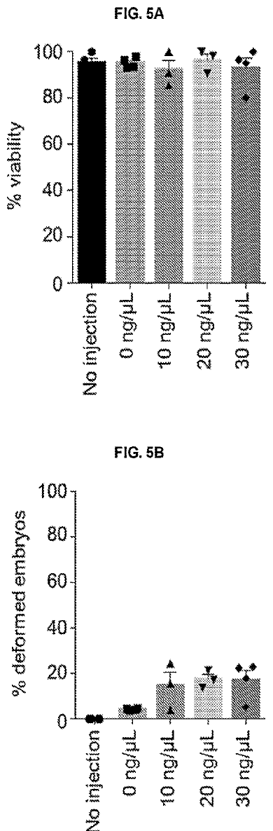

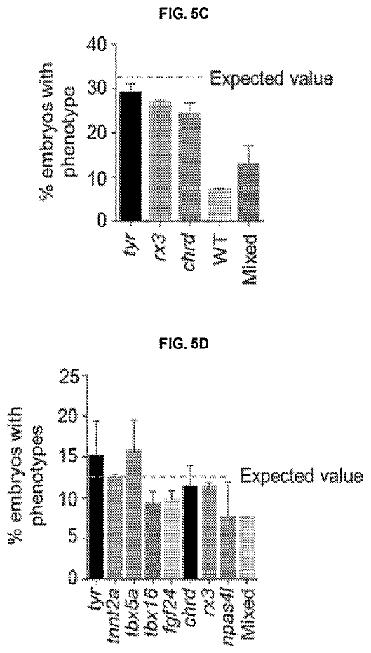

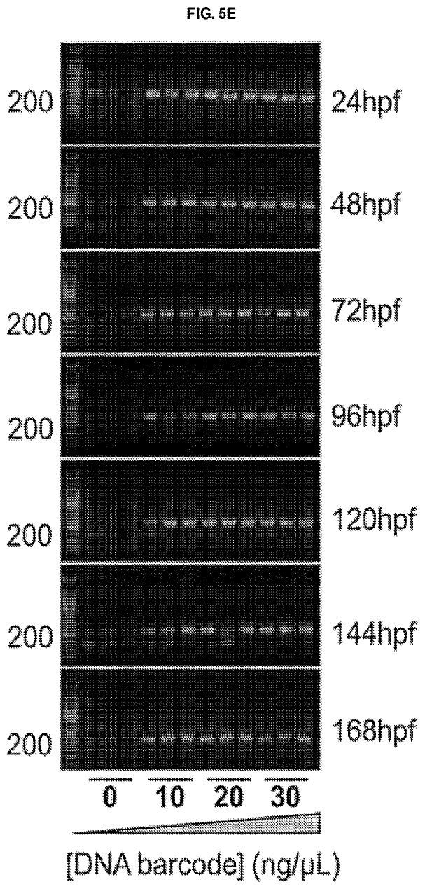

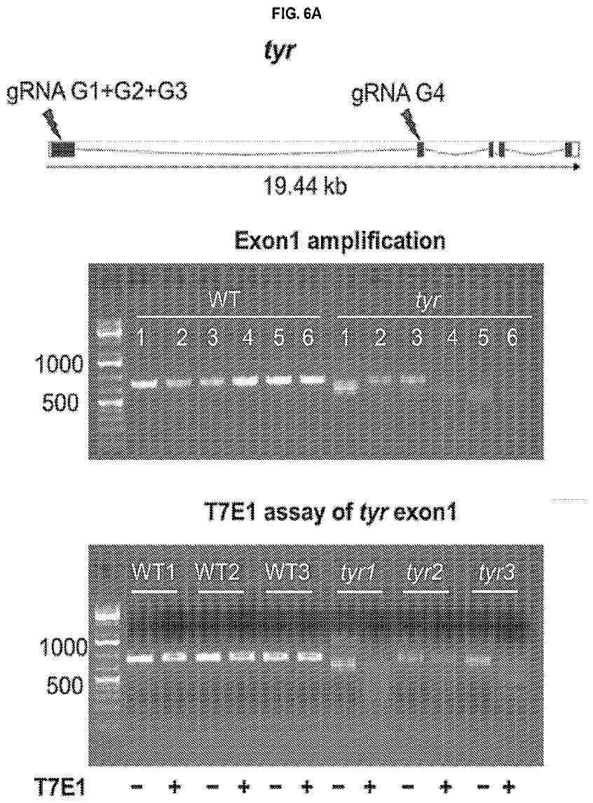

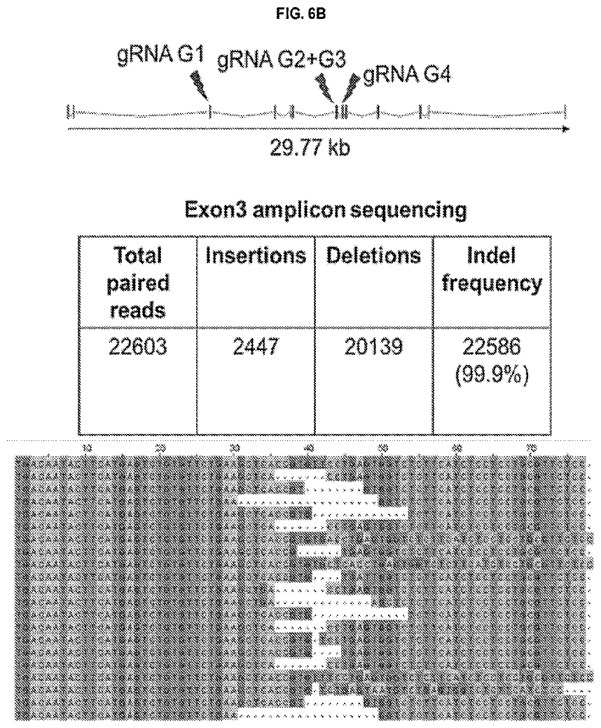

After testing different surfactant-oil combinations, a combination of fluorinated oil and a fluorosurfactant as optimal for droplet generation was identified using a repurposed Bio-Rad QX-200 droplet generator. The droplets generated were uniform, ˜100 um in diameter (FIG. 3B). Each droplet contained four gRNAs targeting a gene-of-interest. It was found that using four gRNAs per gene recapitulated the phenotypes of homozygous mutants in F0 embryos with high penetrance (FIG. 4B-D and TABLE 1). Injection of four gRNAs targeting tyr, tnnt2a, tbx5a, rx3, npas41, chrd, tbx16, and fgf24 resulted in highly efficient biallelic mutagenesis (FIG. 5A-B) and the expected albino, silent heart, stringy heart, eyeless, cloche, tissue ventralization, spadetail, and lack of pectoral fins phenotypes respectively in 70-100% of the F0 embryos. Importantly, no significant toxicity was observed in embryos injected with MIC-Drop compared to traditional RNP injection (FIG. 3C-D and FIG. 6A). Droplets were stable during prolonged storage and showed high phenotypic penetrance even after a month of storage at 4° C. (FIG. 3D). Additionally, injection of intermixed MIC-Drops targeting 3-8 different genes and subsequent phenotyping revealed that most embryos had a unique phenotype demonstrating successful injection of a single droplet per embryo (FIG. 3F and FIG. 5C-D). Importantly, the frequency of each phenotype was close to the expected value, indicating proportionate representation of each droplet within a mixed pool. Finally, the injected DNA barcodes could be recovered at least up to 7 days post fertilization (dpf) (FIG. 5E). Retrieval and sequencing of the barcode from the injected embryos revealed a high genotype-phenotype correlation.

### Example 3

### Sensitivity of MIC-Drop Gene Identification

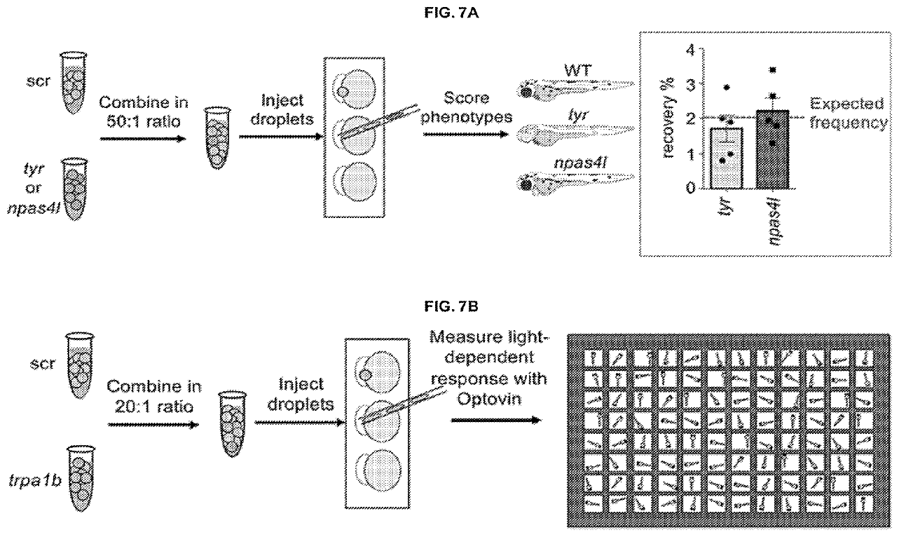

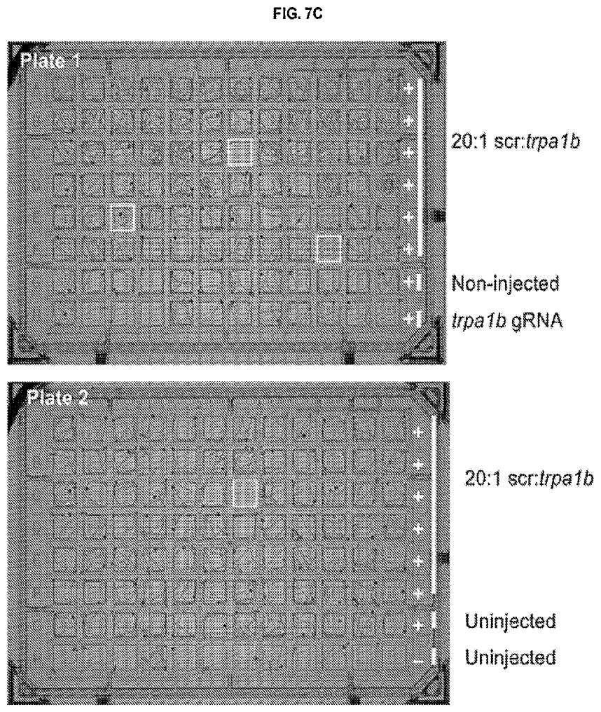

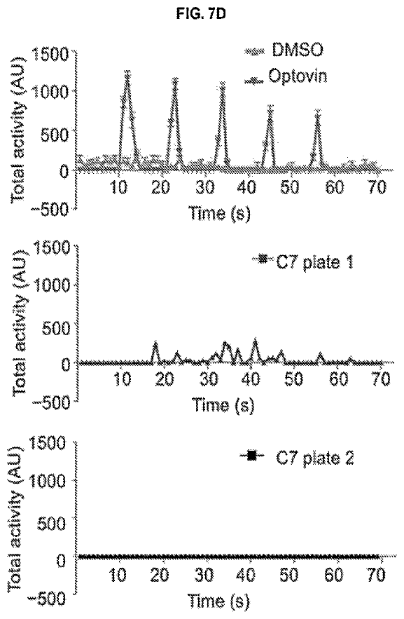

Next, it was tested whether MIC-Drop could identify genes responsible for a particular phenotype from a list of candidate genes (FIG. 7A). Droplets targeting the tyr or npas4/genes were spiked into a larger pool of droplets containing scrambled gRNAs such that the tyr or npas4/MIC-drops each represented 2% of the total. Hundreds of embryos were injected with the intermixed droplets and the frequency of albino and cloche phenotypes among the injected embryos was assessed. Frequencies of (1.7±0.8) % and (2.2±0.8) % for the albino and cloche phenotypes were observed, respectively (FIG. 7A inset), comparable to theoretical expected frequency of 2%, thereby indicating MIC-Drop screens are sensitive and may be a useful platform for a variety of applications requiring identification of genotype-phenotype relationships in vertebrates on a large scale.

### Example 4

### Identifying Targets of Small Molecules Using MIC-Drop

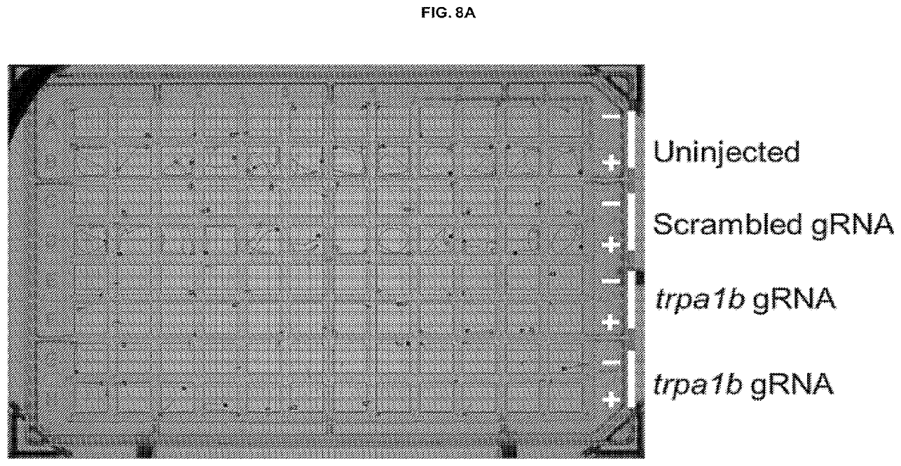

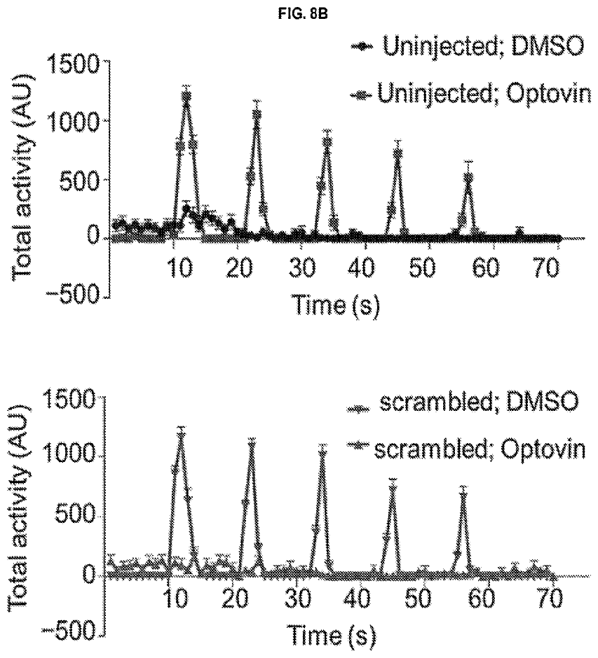

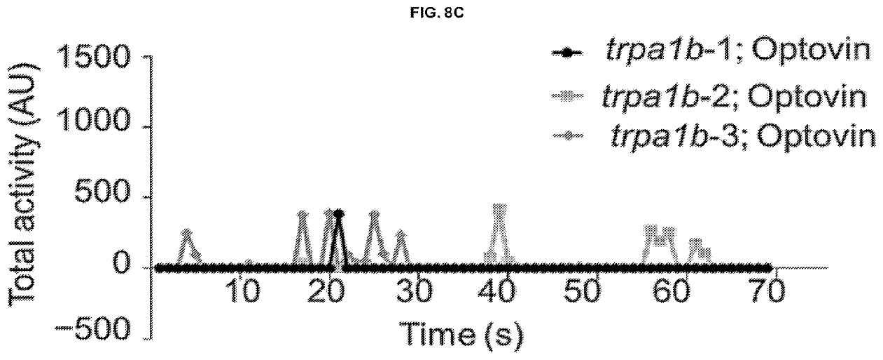

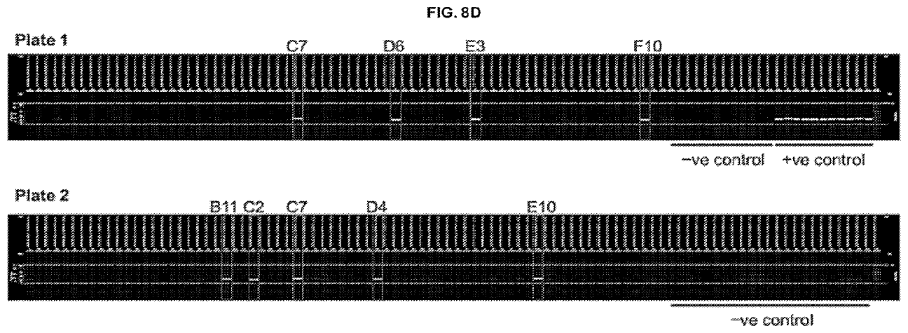

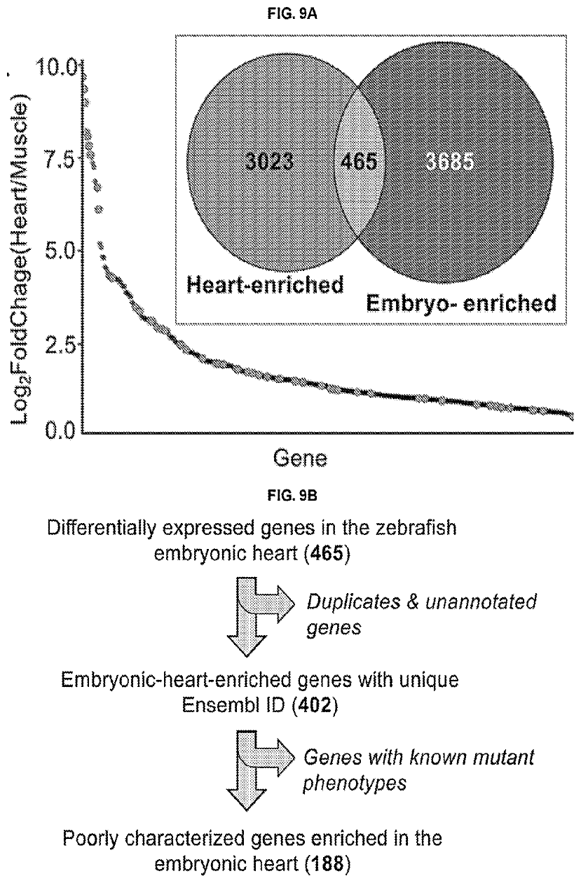

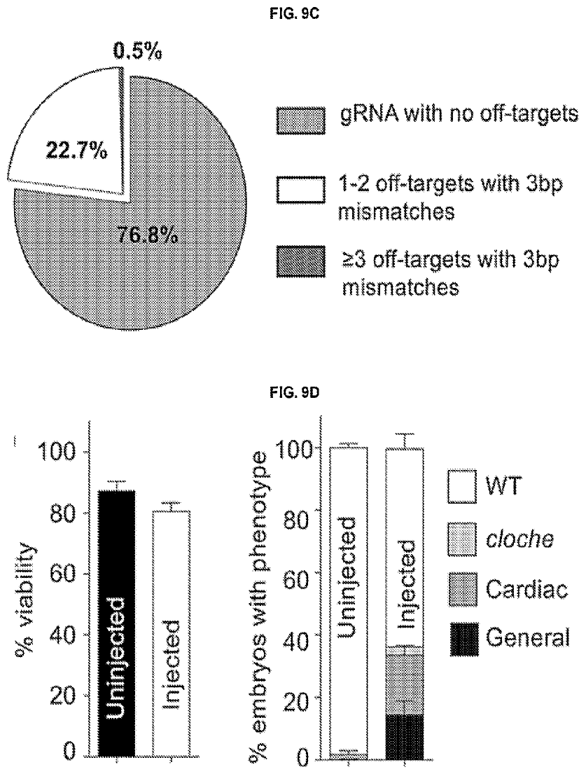

Identifying the protein targets of small molecules remains one of the major challenges in chemical biology and pharmacology. Herein it was hypothesized that MIC-Drop could be used to identify the targets of small molecules that result in complex behavioral phenotypes in the zebrafish. As proof-of-principle, optovin was utilized, a small molecule agonist of the trpa1b channel that allows photo-activatable behavioral modifications in zebrafish. Droplets targeting the trpa1b channel were spiked into a collection of droplets containing scrambled gRNAs in a 1:20 ratio (FIG. 7B). Droplet-injected embryos were arrayed into 96-well plates, treated with optovin and exposed to violet light flashes while simultaneously recording embryo movement. Treatment of wild-type zebrafish embryos with optovin resulted in a light-dependent motor response (FIG. 8A-C). Embryos that showed reduced or no movement in the assay were isolated, and their barcodes sequenced for genotype verification. It was found that 2-3% of embryos showed a complete loss of photo-induced motion (FIG. 7B, FIG. 8D). Barcode sequencing revealed 100% of the unresponsive embryos were of trpa1b genotype. An additional ˜2% of the embryos showed photo-induced motor response despite being of the trpa 1b genotype, likely due to incomplete loss of trpa1b function (FIG. 8D). Thus, the MIC-Drop platform was able to be used to identify the target of optovin from among a library of non-target candidates.

### Example 5

### Identification of Genes Responsible for a Range of Phenotypes Using MIC-Drop

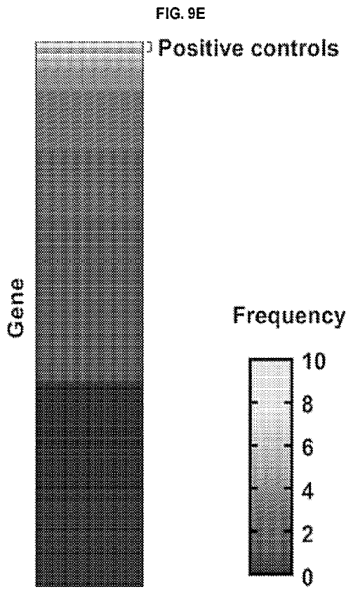

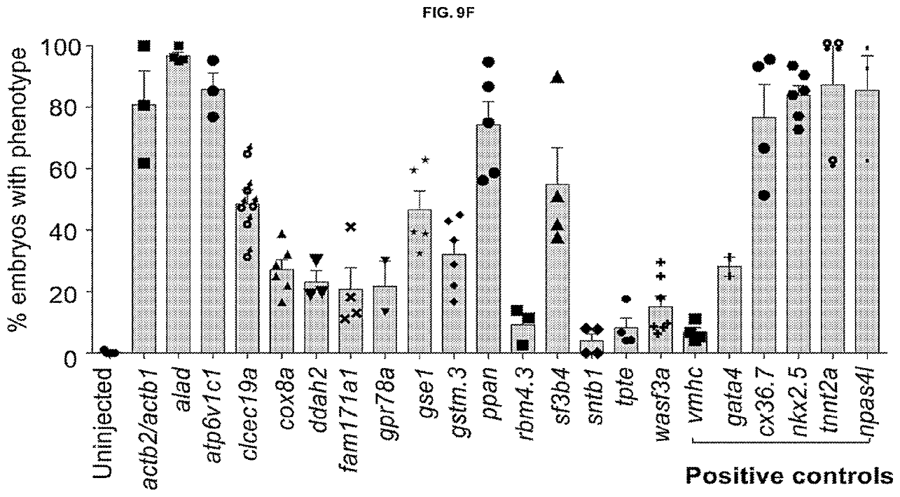

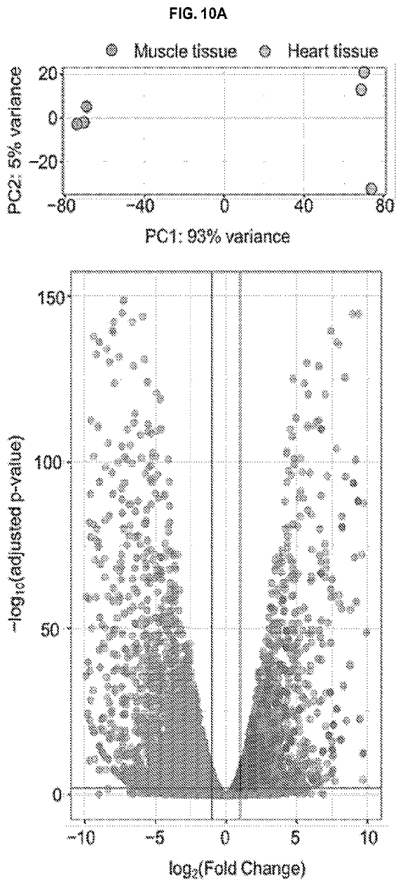

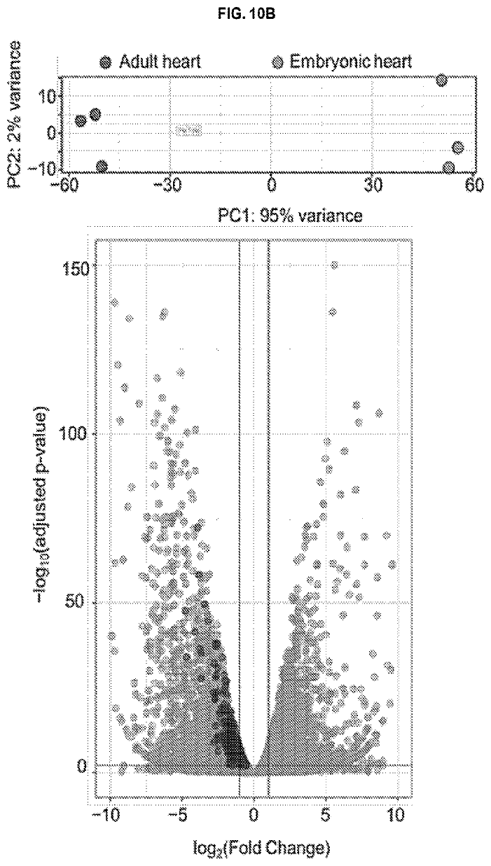

Large-scale forward genetic screens in zebrafish have been highly successful in identifying genes involved in developmental and behavioral phenotypes. However, uncovering the genetic bases for these phenotypes remains a lengthy and laborious process. MIC-Drop can be used to rapidly perform large-scale, reverse-genetic screens to uncover genes responsible for important phenotypes such as developmental defects in the cardiovascular system. Congenital Heart Disease (CHD) is the most common form of birth defect in humans, affecting nearly 1% of all live births. Genetic factors play a strong causal role in the development of CHD, however, a comprehensive understanding of all the genes responsible for CHD is still lacking. Publicly available RNAseq datasets were used to curate a list of 188 poorly characterized genes that are enriched in the zebrafish embryonic heart tissue relative to muscle tissue (FIG. 9A-B, FIG. 10A-B, and Supplementary Tables 2-4 of Parvez et al. (2021) Science. 373:6559, 1146-1151) and it was postulated that these genes might be important in vertebrate heart development. A MIC-drop library containing MIC-drops for all 188 genes, plus several control genes, was generated (FIG. 9C and Supplementary Table 5 of Parvez et al. (2021) Science. 373:6559, 1146-1151). Morphological phenotyping of zebrafish embryos at 48-72 hpf after MIC-Drop injection identified 13 novel genes, the loss of which result in cardiac or blood phenotypes (FIG. 9D-E). Secondary validation of these “hits” corroborated the findings of the initial screen, with 10/13 genes showing phenotypic penetrance in >20% of F0 embryos (FIG. 9E). Interestingly, the screen identified genes responsible for a range of phenotypes including 1 gene (alad) responsible for porphyria, 2 genes (gstm.3 and atp6v1c1) responsible in arrhythmia, and 7 genes (actb2, clec19a, gse1, ppan, sf3b4, cox8a, and ddah2) responsible for normal cardiac development and looping.

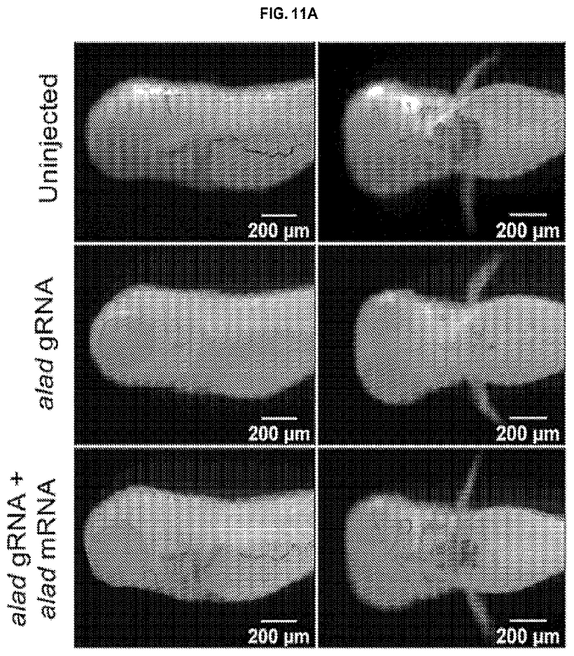

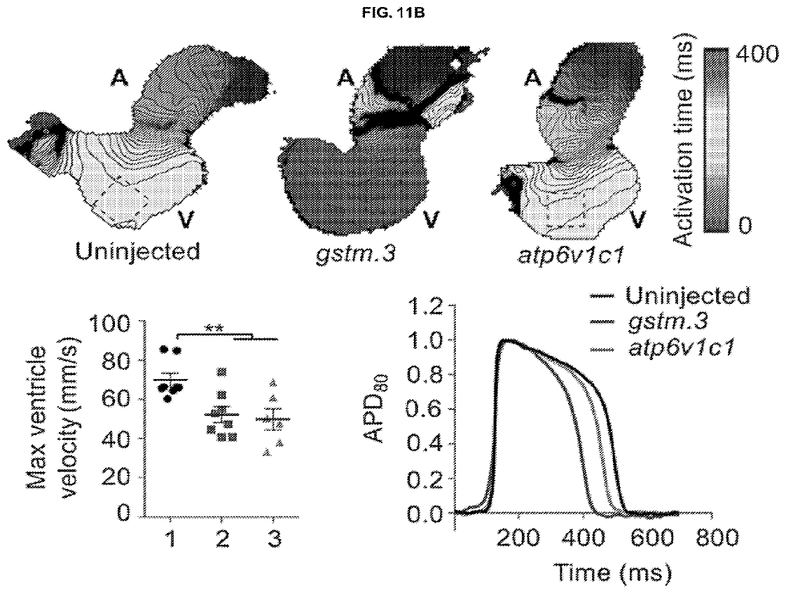

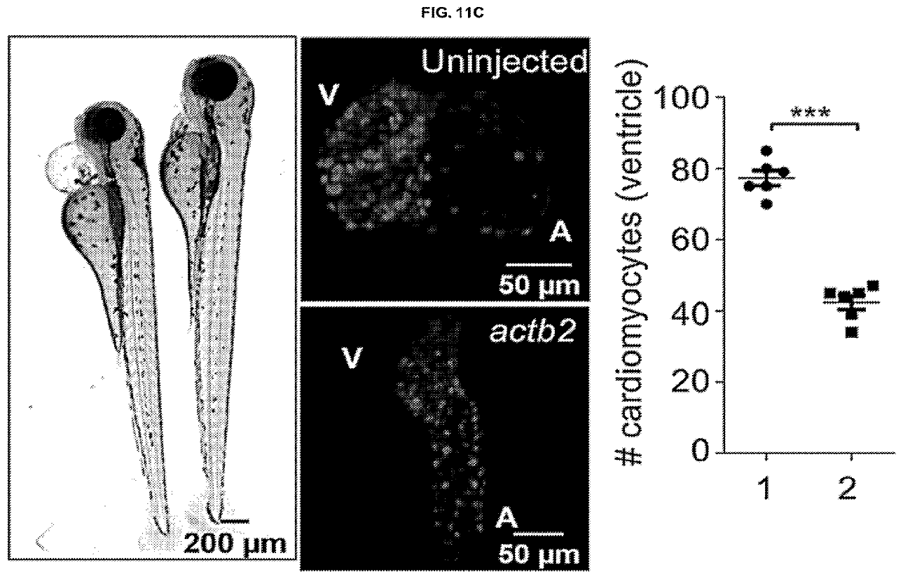

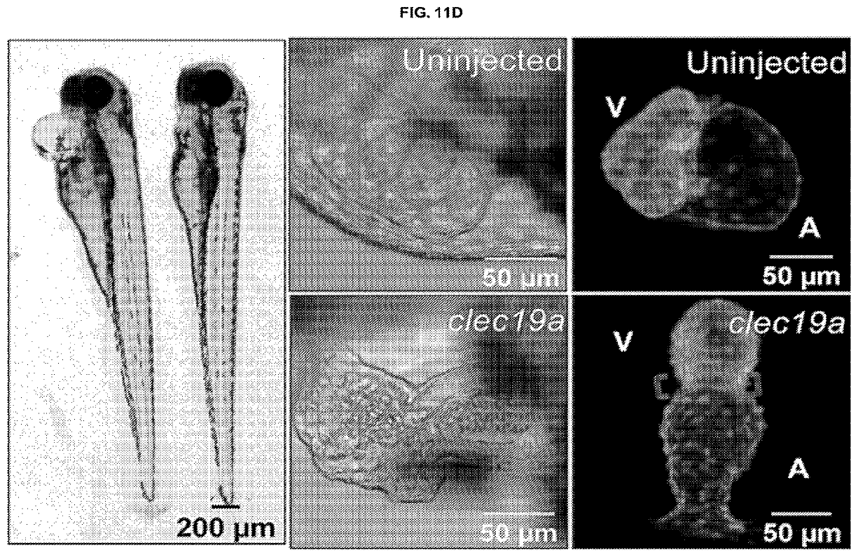

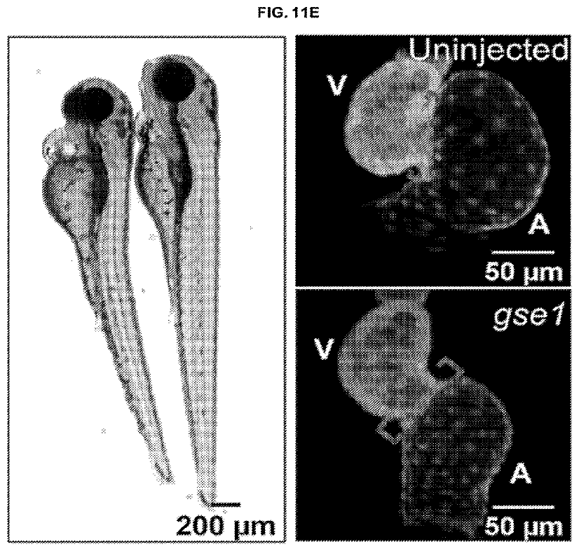

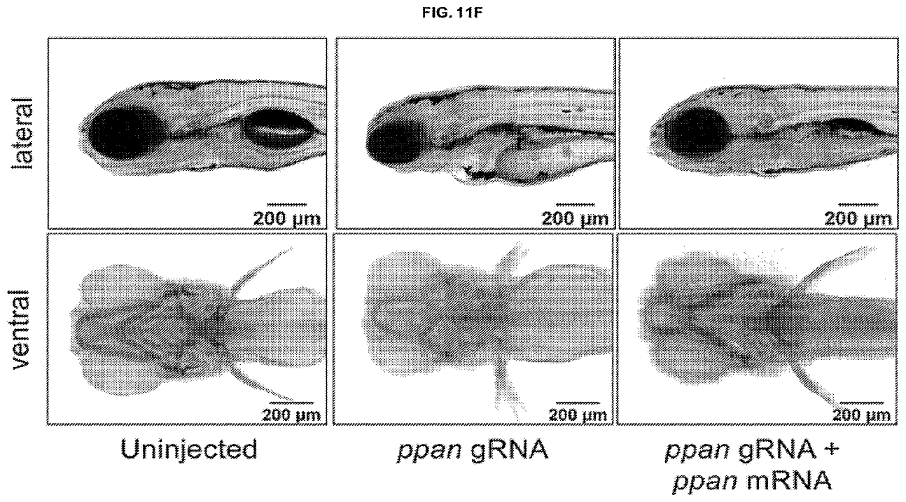

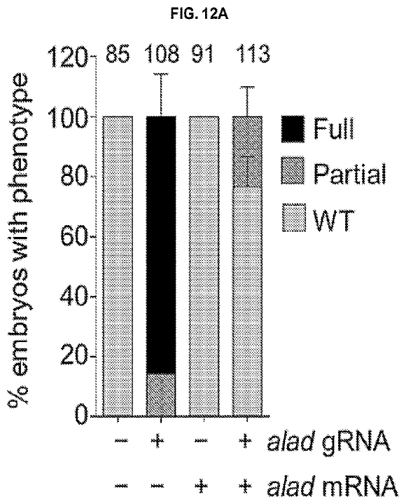

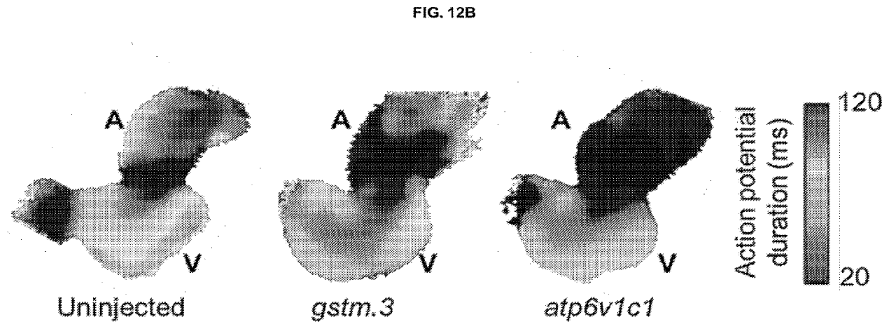

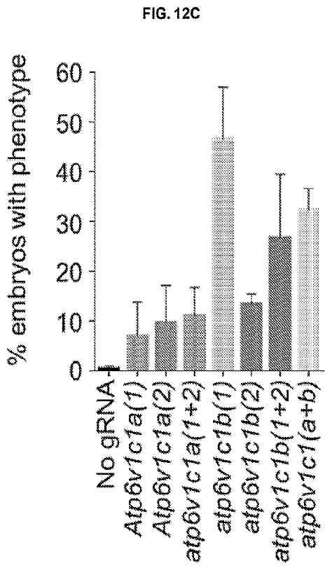

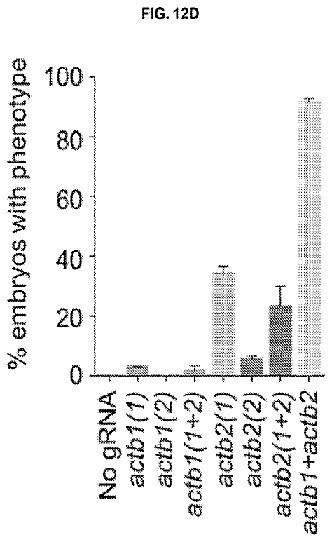

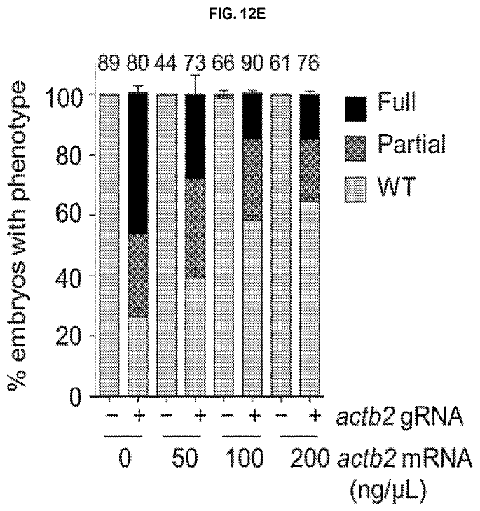

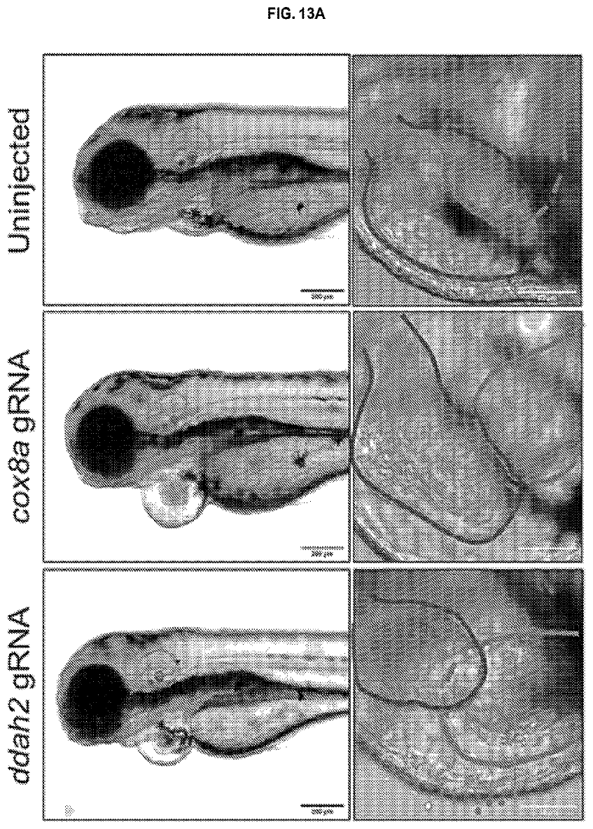

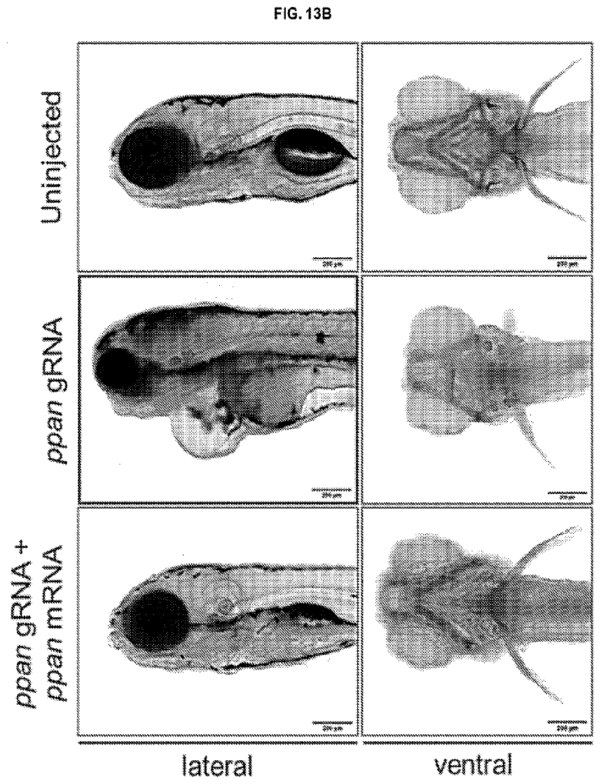

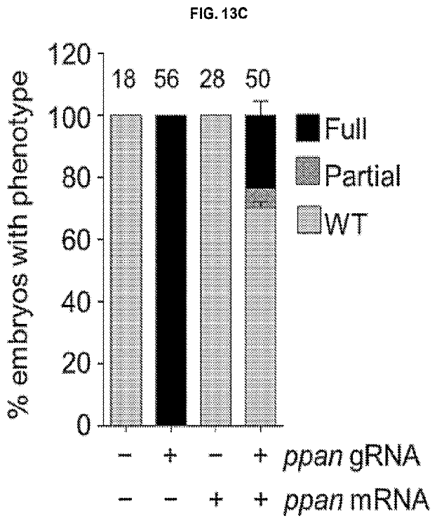

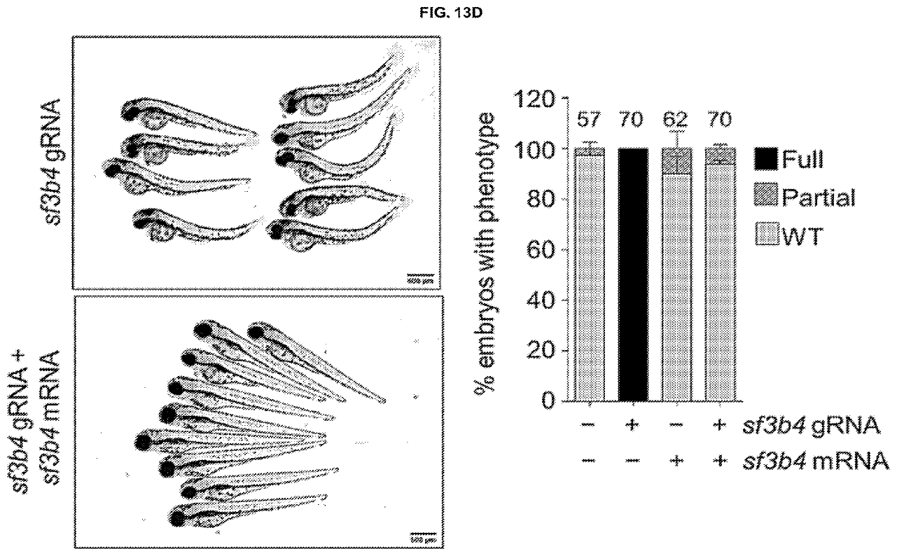

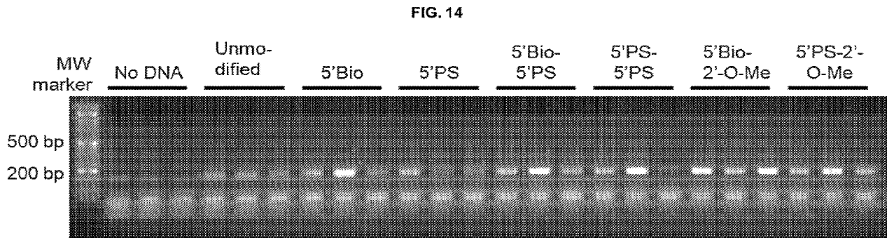

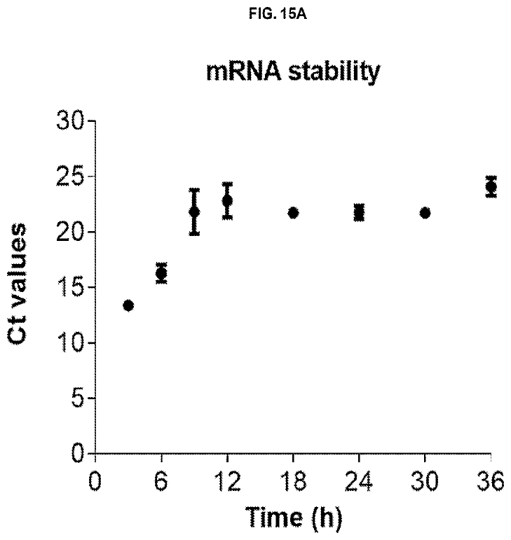

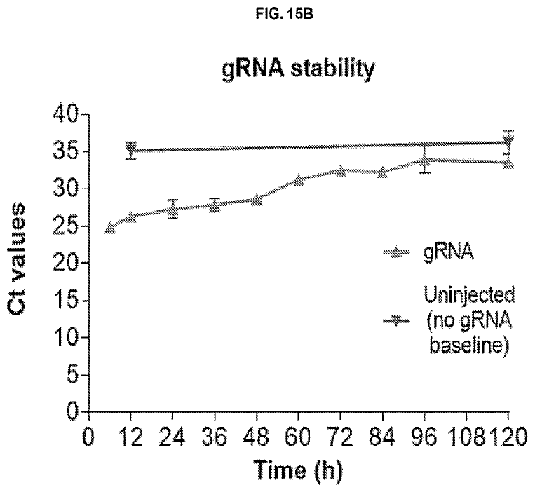

Deeper characterization of the F0 crispant phenotypes was performed. Additionally, to ensure the phenotypes are due to on-target gene knockout, phenotype rescue with mRNA injection was performed. alad crispants showed a complete loss of hemoglobin synthesis which was rescued by injection of alad mRNA (FIG. 11A and FIG. 12A). Voltage mapping of the gstm.3 and atp6v1c1 crispants showed slowed atrial and ventricular conductions and altered action potential duration (FIG. 11B and FIG. 12B). We identified atp6v1c1b as the ohnolog responsible for the ventricular arrhythmia phenotype (FIG. 12C). GSTM3 was recently identified as a risk factor in Brugada syndrome with increased susceptibility to sudden cardiac death. Germline gstm.3 zebrafish mutants exhibited ventricular arrhythmia corroborating the results observed in MIC-Drop crispants. Loss of function of several genes resulted in cardiac development defects. β-actin (actb1 and actb2) crispants showed cardiac edema, a small, silent ventricle with reduced cardiomyocytes, leaky blood vessels as well as gross craniofacial defects (FIG. 11C). Interestingly, loss of actb2 alone was sufficient to recapitulate the cardiac phenotypes without the gross morphological defects suggesting actb2 and actb1 have non-overlapping roles (FIG. 11C and FIG. 12D-E). clec19a, ac-type lectin protein with unknown functions was identified as important for the normal development of cardiac jelly and the atrioventricular valve in 3 dpf zebrafish embryos (FIG. 11D). Additionally, cox8a, a component of the mitochondrial electron transport chain and ddah2, an arginine metabolizing enzyme were shown to be important for normal cardiac function (FIG. 13A). Finally, three other genes with limited annotation of their functions were identified as being important in heart development. Loss of ppan, gse1, and sf3b4 resulted in cardiac abnormalities along with other development defects such as malformed bones/cartilages in the jaw and pharyngeal arches (ppan), bent trunk (gse1 and sf3b4), and craniofacial defects (sf3b4) causing embryonic lethality (FIG. 11E-F and FIG. 13B-D). Overexpression of the corresponding proteins rescued the developmental phenotypes. Therefore, MIC-drop enabled a highly efficient reverse-genetic CRISPR screen in an intact vertebrate, leading to the discovery of several genes that contribute to cardiac development or function.

In conclusion, the microfluidics-based platform as described herein can successfully be used for large-scale CRISPR screens in a vertebrate. CRISPR screens have previously been performed in cultured cells, but genome editing in vertebrates has primarily been done one gene at a time. The few small-scale CRISPR screens reported in vertebrates were enabled by brute force scaling of single-gene methods for generating, tracking, and analyzing individual genes, with little economy of scale. By intermixing droplets targeting many genes and by incorporating a barcode for retrospective target identification, the MIC-drop platform as described herein enables zebrafish to be injected, housed, and analyzed en masse, with rapid identification of the target genes in individuals exhibiting phenotypes of interest. The pilot screen reported here quickly discovered several genes important for cardiovascular development and function. This screen of 188 genes was completed within a few weeks and could readily be scaled to thousands of genes or even to full genome scale. Moreover, MIC-Drop is versatile and conceptually can be used not just for gene knockout but for other screens such as CRISPR activation/inactivation screens and functional screens of non-coding genetic elements. Finally, the platform can be adapted for use in other model organisms including Xenopus and mouse embryos where F0 crispants are shown to recapitulate known germline mutant phenotypes. Thus, the MIC-Drop platform enables in vivo vertebrate CRISPR experiments to be performed with the speed, efficiency, and scale previously only available to in vitro systems.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A water-in-oil droplet comprising: an aqueous phase comprising a gene editing system and a barcode oligonucleotide; and an oil phase comprising an oil and a surfactant; wherein the aqueous phase is encapsulated by the oil phase.

Clause 2. The water-in-oil droplet of clause 1, wherein the gene editing system is a Clustered Regularly Interspaced Short Palindromic Repeats-CRISPR associated proteins (CRISPR-Cas) system, a transcription activator like effector nuclease (TALEN) system, or a zinc finger nuclease (ZFN) system.

Clause 3. The water-in-oil droplet of clause 1 or clause 2, wherein the oil is 3M™ Novec™ 7500, Bio-Rad Droplet Generation Oil for Probes, or a polysiloxane.

Clause 4. The water-in-oil droplet of any one of clauses 1-3, wherein the oil phase comprises from about 90% to about 99.9% of the oil.

Clause 5. The water-in-oil droplet of any one of clauses 1-4, wherein the surfactant is 008-Fluorosurfactant, Pico-Surf™, or a dendronized fluorosurfactant.

Clause 6. The water-in-oil droplet of any one of clauses 1-5, wherein the oil phase comprises from about 0.1% to about 10% of the surfactant.

Clause 7. A method for large-scale identification of a gene in vivo in a plurality of subjects, the method comprising: administering to the plurality of subjects a plurality of barcode oligonucleotides; isolating one or more barcode oligonucleotides from one or more subjects from the plurality of subjects that exhibit one or more phenotypes of interest; amplifying the isolated barcode oligonucleotides; and, sequencing the amplified barcode oligonucleotides.

Clause 8. The method of clause 7, wherein the barcode oligonucleotides comprise an end-cap modification at the 5′ end of the oligonucleotide.

Clause 9. The method of clause 8, wherein the end-cap modification is biotinylation, 2′OMe, or phosphorothioate.

Clause 10. The method of any one of clauses 7-9, wherein the barcode oligonucleotide is unmodified.

Clause 11. The method of any one of clauses 7-10, wherein the plurality of subjects are highly prolific organisms.

Clause 12. The method of clause 11, wherein the highly prolific organisms are fish, insects, or worms.

Clause 13. A method for large-scale identification of gene function in a plurality of subjects, the method comprising: administering to the plurality of subjects a plurality of water-in-oil droplets comprising: an aqueous phase comprising a gene editing system and one or more barcode oligonucleotides; and an oil phase, wherein the aqueous phase is encapsulated by the oil phase; isolating the one or more barcode oligonucleotides from one or more subjects from the plurality of subjects that exhibit one or more phenotypes of interest; amplifying the isolated one or more barcode oligonucleotides; and, sequencing the amplified one or more barcode oligonucleotides.

Clause 14. The method of clause 13, wherein the oil phase comprises an oil and a surfactant.

Clause 15. The method of clause 14, wherein the oil is 3M™ Novec™ 7500, Bio-Rad Droplet Generation Oil for Probes, or a polysiloxane.

Clause 16. The method of clause 14 or clause 15, wherein the oil phase comprises from about 90% to about 99.9% of the oil.

Clause 17. The method of any one of clauses 14-16, wherein the surfactant is 008-Fluorosurfactant, Pico-Surf™, or a dendronized fluorosurfactant.

Clause 18. The method of any one of clauses 14-17, wherein the oil phase comprises from about 0.1% to about 10% of the surfactant.

Clause 19. The method of any one of clauses 13-18, wherein the gene editing system is a Clustered Regularly Interspaced Short Palindromic Repeats-CRISPR associated proteins (CRISPR-Cas) system, a transcription activator like effector nuclease (TALEN) system, or a zinc finger nuclease (ZFN) system.

Clause 20. The method of any one of clauses 13-19, wherein the one or more barcode oligonucleotides comprise an end-cap modification at the 5′ end of the oligonucleotide that prevents exonuclease and endonuclease degradation of the one or more barcode oligonucleotides.

Clause 21. The method of any one of clauses 13-20, wherein each subject of the plurality of subjects is administered one water-in-oil droplet from the plurality of water-in-oil droplets that comprises a gene editing system that targets a different gene in each subject.

Clause 22. The method of any one of clauses 13-21, wherein the plurality of water-in-oil droplets are administered to the plurality of subjects simultaneously.

1. A water-in-oil droplet comprising:
an aqueous phase comprising a gene editing system and a barcode oligonucleotide; and
an oil phase comprising an oil and a surfactant;
wherein the aqueous phase is encapsulated by the oil phase.

2. The water-in-oil droplet of claim 1, wherein the gene editing system is a Clustered Regularly Interspaced Short Palindromic Repeats-CRISPR associated proteins (CRISPR-Cas) system, a transcription activator like effector nuclease (TALEN) system, or a zinc finger nuclease (ZFN) system.

3. The water-in-oil droplet of claim 1, wherein the oil is 3M™ Novec™ 7500, Bio-Rad Droplet Generation Oil for Probes, or a polysiloxane.

4. The water-in-oil droplet of claim 1, wherein the oil phase comprises from about 90% to about 99.9% of the oil.

5. The water-in-oil droplet of claim 1, wherein the surfactant is 008-Fluorosurfactant, Pico-Surf™, or a dendronized fluorosurfactant.

6. The water-in-oil droplet of claim 1, wherein the oil phase comprises from about 0.1% to about 10% of the surfactant.

7. A method for large-scale identification of a gene in vivo in a plurality of subjects, the method comprising:
administering to the plurality of subjects a plurality of barcode oligonucleotides;
isolating one or more barcode oligonucleotides from one or more subjects from the plurality of subjects that exhibit one or more phenotypes of interest;
amplifying the isolated barcode oligonucleotides; and,
sequencing the amplified barcode oligonucleotides.

8. The method of claim 7, wherein the barcode oligonucleotides comprise an end-cap modification at the 5′ end of the oligonucleotide.

9. The method of claim 8, wherein the end-cap modification is biotinylation, 2′OMe, or phosphorothioate.

10. The method of claim 7, wherein the barcode oligonucleotide is unmodified.

11. The method of claim 7, wherein the plurality of subjects are highly prolific organisms.

12. The method of claim 11, wherein the highly prolific organisms are fish, insects, or worms.

13. A method for large-scale identification of gene function in a plurality of subjects, the method comprising:
administering to the plurality of subjects a plurality of water-in-oil droplets comprising:
an aqueous phase comprising a gene editing system and one or more barcode oligonucleotides; and
an oil phase, wherein the aqueous phase is encapsulated by the oil phase;
isolating the one or more barcode oligonucleotides from one or more subjects from the plurality of subjects that exhibit one or more phenotypes of interest;
amplifying the isolated one or more barcode oligonucleotides; and,
sequencing the amplified one or more barcode oligonucleotides.

14. The method of claim 13, wherein the oil phase comprises an oil and a surfactant.

15. The method of claim 14, wherein the oil is 3M™ Novec™ 7500, Bio-Rad Droplet Generation Oil for Probes, or a polysiloxane.

16. The method of claim 14, wherein the oil phase comprises from about 90% to about 99.9% of the oil.

17. The method of claim 14, wherein the surfactant is 008-Fluorosurfactant, Pico-Surf™, or a dendronized fluorosurfactant.

18. The method of claim 14, wherein the oil phase comprises from about 0.1% to about 10% of the surfactant.

19. The method of claim 13, wherein the gene editing system is a Clustered Regularly Interspaced Short Palindromic Repeats-CRISPR associated proteins (CRISPR-Cas) system, a transcription activator like effector nuclease (TALEN) system, or a zinc finger nuclease (ZFN) system.

20. The method of claim 13, wherein the one or more barcode oligonucleotides comprise an end-cap modification at the 5′ end of the oligonucleotide that prevents exonuclease and endonuclease degradation of the one or more barcode oligonucleotides.

21. The method of claim 13, wherein each subject of the plurality of subjects is administered one water-in-oil droplet from the plurality of water-in-oil droplets that comprises a gene editing system that targets a different gene in each subject.

22. The method of claim 13, wherein the plurality of water-in-oil droplets are administered to the plurality of subjects simultaneously.
